(19) United States
(12) Patent Application Publication
(10) Pub. No.: US 2010/0127067 A1
(43) Pub. Date: May 27, 2010

(54) PRINT DEVICE FOR SPECIMEN TRACKING AND MANAGEMENT

(76) Inventors: Peter M. Eisenberg, Minneapolis, MN (US); Mitchell B. Grunes, Minneapolis, MN (US)

Correspondence Address: 3M INNOVATIVE PROPERTIES COMPANY, PO BOX 33427, ST. PAUL, MN, 55133-3427, US

(21) Appl. No.: 12/529,823

(22) PCT Filed: Feb. 27, 2008
(86) PCT No.: PCT/US2008/055120

§ 371 Date: Sep. 3, 2009

Related U.S. Application Data
(60) Provisional application No. 61/066,120, filed on Mar. 8, 2007.

(51) Int. Cl. G06F 17/00; G06K 19/00

(52) U.S. Cl. 235/375; 235/492

(57) ABSTRACT

Techniques are described for using radio-frequency identification (RFID) tags to track patient specimens throughout the collection and analysis of patient specimens. A series of RFID tags may be used to track the specimens starting with the collection of a patient's tissue specimen at a surgery or examination room, through the process of preparing the specimens at a laboratory, to a specialist's office where the specimens are analyzed, and eventually into storage where the left-over specimen materials may be archived. A database may be used to capture unique identifiers for the RFID tags and other information throughout the process.

### TECHNICAL FIELD

The invention relates to anatomic pathology specimens, and more particularly, to techniques for managing anatomic pathology specimens.

### BACKGROUND

Hospitals and clinics routinely collect biological specimens from patients, and analyze the specimens to diagnose diseases. For example, a surgeon may perform a biopsy of a tumor to extract a biopsy specimen, and a pathologist analyzes the biopsy specimen to determine whether the tumor is benign or malignant. During the process of collection, preparation of the specimen, and analysis, a single specimen undergoes numerous hand-offs between individuals, departments, and even different institutions. At each location, the specimen may be split into several constituent samples.

For example, a specimen from a patient may initially be placed in one or more labeled containers such as bottles. The bottles are typically then sent to an anatomic pathology lab, where the tissue may be cut and placed into labeled cassettes. Tissue from a single bottle may, for example, be divided into multiple cassettes. The tissue may then be dehydrated and embedded in wax to form a block. Next, one or more slides may then be prepared using tissue from a single specimen block. In particular, thin sections of the specimen block are shaved and placed on different labeled slides. The slides are stained and slip covers are added. The slides are then transferred from the lab to a pathologist's office, where the pathologist analyzes the slides and creates a pathology report that is added to the patient's record. Results of the pathology report are communicated to the patient. The remaining slides, blocks, or bottles may be archived.

Proper handling of patient-specific specimens is potentially one of the most important aspects of a specimen analysis process. Errors in the processing of the specimen can result in failures ranging from delays in processing and analysis, incorrect information being provided to a patient, and even harm to the patient. Such errors may even give rise to malpractice lawsuits. It is, therefore, important to properly identify each bottle, block, and slide.

### SUMMARY

In general, the invention relates to techniques for using radio-frequency identification (RFID) tags to manage patient-specific material throughout the entire process of collection, preparation, and analysis of anatomic pathology specimens. A series of RFID tags may be used to manage the patient-specific material starting with the collection of specimens from a patient at a hospital, through processing the specimens at a laboratory facility, to analysis of the specimens by a pathologist, and eventually into storage where materials may be archived.

A specimen management system includes RFID stations deployed throughout an institution, and possibly multiple institutions, to track and manage the patient-specific material throughout the entire process. The RFID stations may maintain one or more databases to store patient records, where each patient record includes patient information and unique identifiers for the RFID tags associated with the anatomic pathology specimens for the patient. Moreover, the specimen management system may interface with other systems, such as patient management systems and laboratory information systems used during the process.

Various techniques are employed to ensure that the materials and specimens are associated with the proper patient. For example, for a given case, the specimen management system may require that digital information is transferred between RFID tags or stored within the central database only upon verifying that the proper specimens are present and accounted for at any particular location within the process.

For example, when multiple tissue blocks are prepared from a single tissue specimen contained with a particular bottle, the specimen management system may verify that the RFID tag for the bottle as well as all of the RFID tags assigned to the tissue blocks are present and within communication range before transferring digital information from the RFID tag of the bottle to the series of RFID tags for the blocks or otherwise recording successful preparation of the blocks. Similarly, when multiple slides are prepared with tissue from a single tissue block, the specimen management system may require that the RFID tag for the tissue block as well an RFID tag of a slide is present and within communication range before transferring digital information from the RFID tag of the block to the RFID tag of the slide. In this manner, the specimen management system may help ensure that patient-specific materials are associated with the proper patient records and that no materials are lost or incorrectly labeled at any point within the process.

In one embodiment, a method comprises positioning a first anatomical pathology specimen container within a range of a radio-frequency identification (RFID) antenna located within a medical facility, positioning a second anatomical pathology specimen container within the range of the RFID antenna, wherein the first and the second anatomical pathology specimen containers are each associated with respective RFID tags, and wherein the second anatomical pathology specimen container holds a specimen that is at least a portion of a specimen formerly held by the first anatomical pathology specimen container, reading information from the RFID tag of the first anatomical pathology specimen container, determining, based on the information from the RFID tag of the first anatomical pathology specimen container, a patient record maintained by a database of a specimen management system, reading information from the patient record maintained by the database of the specimen management system, and programming the RFID tag of the second anatomical pathology specimen container with the information read from the patient record.

In a further embodiment, a system comprises a first anatomical pathology specimen container having a radio frequency identification (RFID) tag, and a second anatomical pathology specimen container having an RFID tag, wherein the second anatomical pathology specimen container holds a specimen that is at least a portion of a specimen formerly held by the first anatomical pathology specimen container. The system further comprises a specimen management system that maintains a patient record and a radio-frequency identification (RFID) antenna located within a medical facility, wherein the RFID antenna is configured to read information from the patient record maintained by the database of the specimen management system and program the RFID tag of the second anatomical pathology specimen container with the information read from the patient record.

In another embodiment, a method comprises receiving a set of anatomical pathology specimen containers at a location of a medical facility, each of the anatomical pathology specimen containers having an RFID tag, and interrogating the RFID tags of each of the anatomical pathology specimen containers to determine whether the received anatomical pathology specimen containers represent a complete set of anatomical pathology specimen containers associated with a source specimen obtained from a patient.

In a further embodiment, a system comprises a radio-frequency identification (RFID) station comprising an RFID reader and an RFID antenna, and a specimen management system in communication with the RFID reader, wherein when the RFID station receives a set of anatomical pathology specimen containers each having a radio-frequency identification (RFID) tag, the RFID antenna interrogates the RFID tags of each of the RFID tags of each of the anatomical pathology specimen containers, and the RFID reader accesses the specimen management system to determine whether the received anatomical pathology specimen containers represent a complete set of anatomical pathology specimen containers associated with a source specimen obtained from a patient.

In yet another embodiment, a printer comprises a print output for printing label information to a label, a radio-frequency identification (RFID) encoder embedded within the printer for programming an RFID tag associated with the label produced by the print output, wherein the RFID encoder includes an RFID reader and a first RFID antenna, and a second RFID antenna coupled to the RFID reader for reading information from the RFID tag, wherein the reader verifies accuracy of information programmed to the RFID tag on the label based on the information read from the RFID tag.

In one embodiment, a method comprises printing label information to a label with a print output of a printer, programming a radio-frequency identification (RFID) tag associated with the label produced by the print output with an RFID encoder embedded within the printer, reading the RFID tag with a second RFID antenna coupled to the RFID reader, and verifying the accuracy of information programmed to the RFID tag on the label based on the information read from the RFID tag.

In a further embodiment, a method comprises collecting one or more tissue specimens from a patient at a surgical location within a medical facility, programming, at the surgical location, radio frequency identification (RFID) tags associated with a set of anatomical pathology specimen containers that contain the one or more tissue specimens, associating identifiers of the RFID tags with a patient record associated with the patient in a first information management system, receiving one or more of the set of anatomical pathology specimen containers at a laboratory, interrogating, at the laboratory, at least one RFID tag of the anatomical pathology specimen containers to obtain identification information, retrieving information for the patient from the first information management system using the identification information, and storing the patient information in a laboratory information system.

In yet another embodiment, a system comprises a radio frequency identification (RFID) station within a surgery room for collecting one or more tissue specimens from a patient, the RFID station within the surgery room configured to program RFID tags associated with a set of anatomical pathology specimen containers that contain the one or more tissue specimens and associate identifiers of the RFID tags with a patient record associated with the patient in a first information management system, and an RFID station within a laboratory for receiving one or more of the set of anatomical pathology specimen containers and processing the tissue specimens, the RFID station within the laboratory configured to interrogate at least one RFID tag of the anatomical pathology specimen containers to obtain identification information, retrieve information for the patient from the first information management system using the identification information, and store the patient information in a laboratory information system

In another embodiment, a method comprises configuring a set of one or more rules within a specimen management system to define a route for an anatomical pathology specimen container having a radio frequency identification (RFID) tag, wherein the route includes two or more expected locations having RFID readers, and wherein the set of rules includes an expected time period for the anatomical pathology specimen container to travel between two of the expected locations on the route. The method further comprises interrogating the RFID tag of the anatomical pathology specimen container with an RFID reader upon receiving the anatomical pathology specimen container at a location, and storing a timestamp to the specimen management system to indicate the time and location of the anatomical pathology specimen container based on the interrogation.

In yet another embodiment, a system comprises a specimen management system having a set of one or more rules, wherein the specimen management system presents a user interface to configure the set of rules to define a route for an anatomical pathology specimen container having a radio frequency identification (RFID) tag, wherein the route includes at least two locations within a medical facility. The system further comprises an RFID station at each of the locations to interrogate the RFID tag of the anatomical pathology specimen container at the respective location, wherein the specimen management system applies the rules to automatically provide an alert upon determining, based on the interrogation of the RFIG tag at one or more of the locations, that shipment of the anatomical pathology specimen container within the medical facility fails to comply with the route defined by the set of rules.

In another embodiment, a method comprises configuring a set of one or more rules to define a time period between association of an anatomical pathology specimen container of a first type with a patient record within a specimen management system and association of an anatomical pathology specimen container of a second type with the patient record, wherein the anatomical pathology specimen container of the second type hold a specimen that is at least a portion of a specimen previously held by the anatomical pathology specimen container of the first type. The method further comprises providing an alert when the specimen management system detects that the anatomical pathology specimen container of a second type has not been associated with the patient record within the time period.

In a further embodiment, a system comprises an anatomical pathology specimen container of a first type having a radio frequency identification (RFID) tag, and an anatomical pathology specimen container of a second type having an RFID tag, wherein the set of anatomical pathology specimen container of the second type holds a specimen that is at least a portion of a specimen previously held by the anatomical pathology specimen container of the first type. The system further comprises a specimen management system having a set of one or more rules, wherein the specimen management system presents a user interface to configure the set of rules to define a time period in which the RFID tag for the anatomical pathology specimen container of the second type is expected to be programmed using information associated with the RFID tag for the anatomical pathology specimen container of the first type.

In another embodiment, a method comprises configuring a set of one or more rules within a specimen management system to define an expected sequence of actions to be performed with respect to a type of tissue specimen, interrogating, at different locations within a medical facility, to retrieve information from a radio frequency identification (RFID) tag of an anatomical pathology specimen container that contains a tissue specimen corresponding to the type of tissue specimen, storing the information for each of the interrogations to a patient record associated with the tissue specimen within the specimen management system, and providing an alert when the specimen management system determines, based on the information, that one or more actions within the expected sequence of actions has failed to occur.

In yet another embodiment, a method comprises checking in a patient at a patient intake location, associating the patient with an examination room, storing the association within an information management system, providing the patient with a patient identification article having a radio frequency identification (RFID) tag, receiving the patient at an examination room, interrogating the RFID tag of the patient identification article at the examination room to obtain patient identification information, accessing the information management system using the patient identification information, and providing an alert when the examination room at which the patient is received does not correspond to the examination room with which the patient was associated.

In a further embodiment, a microscope comprises a stage for mounting a slide to be viewed, wherein the slide is associated with a radio frequency identification (RFID) tag, a lens for magnifying the slide, an eyepiece for viewing a magnified view of the slide, an RFID reader for interrogating the RFID tag of the slide when the slide is mounted on the stage.

In another embodiment, a system comprises a microscope having a stage for mounting a slide associated with a radio frequency identification (RFID) tag, a lens for magnifying the slide, and an RFID reader for interrogating the RFID tag of the slide when the slide is mounted on the stage, and a client computing device in communication with the microscope having a display that displays in a single view a magnified view of the slide obtained from the microscope and patient data obtained from an information management system.

In one embodiment, a method comprises sequentially presenting each of a set of objects to an RFID reader, wherein each of the objects is associated with an RFID tag, interrogating the RFID tags of each of the objects with the RFID reader to obtain information relating to the objects; recalling the information relating to an object for a time period after the object is removed, and verifying that the set of objects is a complete set of objects based on the recalled information.

The techniques of the invention may provide one or more advantages. For example, the techniques may improve tracking and management of anatomic pathology specimens. As another example, the techniques may aid in avoiding errors such as misplaced bottles, blocks, and slides, or a specimen being mistakenly associated with the wrong patient.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an exemplary network environment in which radio frequency identification (RFID) techniques are used for managing patient-specific materials throughout a specimen collection and analysis process.

FIG. 2 is a block diagram illustrating a simplified view of a network environment within a healthcare facility.

FIG. 3 is a flowchart illustrating an exemplary process performed by a healthcare facility in utilizing the RFID specimen management and tracking techniques described herein.

FIG. 4 is a flowchart illustrating in further detail an example process in which RFID tags are utilized during patient intake.

FIG. 5 is a flowchart illustrating in further detail an example process in which RFID tags and a specimen management system are used during specimen collection.

FIGS. 6A-6B are screen illustrations illustrating an example user interface of the specimen management system for programming specimen bottles.

FIG. 7 is a flowchart illustrating in further detail an example process in which RFID tags and a specimen management system are used during laboratory processing.

FIG. 8A is a block diagram illustrating an example specimen block having an RFID tag affixed thereto.

FIG. 8B is a block diagram illustrating an example specimen slide having an RFID tag affixed thereto.

FIG. 8C is a block diagram illustrating an example system for programming RFID tags on specimen blocks using an RFID tag on a specimen bottle.

FIG. 8D is a block diagram illustrating an example system for programming RFID tags on specimen slides using an RFID tag on a specimen block.

FIGS. 8E-8F are block diagrams illustrating example RFID stations used for verifying accuracy of items for a patient case.

FIGS. 9A-9B are screen illustrations illustrating an example user interface of the specimen management system for programming RFID tags on specimen blocks from an RFID tag on a specimen bottle.

FIG. 10 is a flowchart illustrating in further detail an example process in which RFID tags and a specimen management system are used during pathologist analysis of patient specimens.

FIG. 11 is a flowchart illustrating in further detail an example process in which RFID tags and a specimen management system are used during archival of patient specimens.

FIG. 12 is a flowchart illustrating in further detail an example process for verification of accuracy of specimen information by the specimen management system.

FIGS. 13A-13C are screen illustrations illustrating an example user interface of the specimen management system for verification of accuracy of specimen information.

FIG. 14 is an example specimen management system case entry in a specimen management system database.

FIG. 15 is a block diagram illustrating a printer that combines printing of label information and programming RFID information, as well as verifying that the programmed RFID information is correct.

FIG. 16 is a block diagram illustrating an example system having a microscope with an RFID reader for reading an RFID tag associated with a specimen slide.

### DETAILED DESCRIPTION

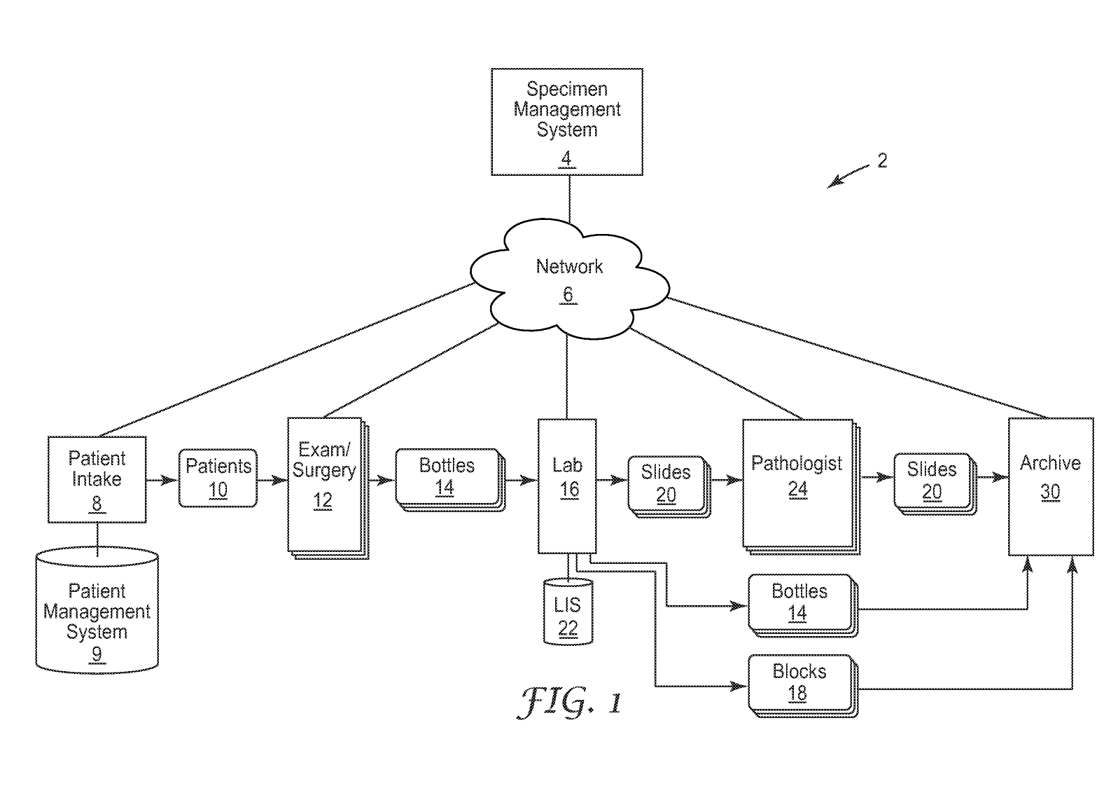

FIG. 1 is a block diagram illustrating an exemplary network environment 2 in which radio frequency identification (RFID) techniques are used for managing patient-specific materials throughout a specimen collection and analysis process. Network environment 2 may be located within a single institution, such as a large hospital, clinic or other health-care facility. Alternatively, network environment 2 may span multiple institutions.

In the example of FIG. 1, network environment 2 includes a specimen management system (SMS) 4 accessible via network 6 to a variety of geographically distributed locations. SMS 4 may be an information management system. As described in further detail below, a heath-care facility that employs SMS 4 may utilize RFID tags to help ensure proper tracking of patient-specific materials during the process of specimen collection and analysis.

For example, RFID tags may be affixed to, embedded within, or otherwise associated with wristbands, containers such as specimen bottles, tissue blocks, pathology slides, archive boxes or other physical objects within environment 2 to track tissue specimens patient 10 throughout the entire collection, preparation and analysis process. SMS 4 stores digital information that defines patient records. Each patent record is typically associated with a different patient and may specify one or more different cases for the patient. Each case may, for example, represent a different visit to the health-care facility by the patient and specifies a set of unique identifiers for RFID tags for anatomic pathology specimens associated with the patient's visit.

Network environment 2 may utilize RFID readers to read and program the RFID tags as the specimens travel from the examination location or surgery 12 to a laboratory 16, to a pathologist 24, and finally to an archive 30. At each different location within in the process, RFID stations at those locations read the RFID tag associated with the specimen to update a status and record timestamps within the RFID tag itself, a database of specimen management system 4, or both.

Initially, patients 10 arrive at a healthcare facility, e.g., a hospital, clinic or other institution, and are checked in at a patient intake site 8 using a patient management system 9. For example, a receptionist may access information about a new patient 10 in the patient management system 9, update any patient information within the patient management system, and record the fact that the patient 10 has been checked in. Patient management system 9 may be an information management system. At this time, the patient 10 may receive a patient identification wristband having an embedded RFID tag and information within specimen management system 4 is synchronized with information within patient management system 9. For example, a patient record within specimen management system 4 may be updated within a unique identifier of the RFID tag of the patent identification wristband as well as identification information (e.g., a patient identifier) that uniquely identifies the patient information within patient management system 9. In addition, or alternatively, the RFID tag within the patient identification wristband may be programmed to store the patient identification information of patient management system 9.

After initial processing, the patient 10 is typically transferred to an examination location or surgery room 12, where a practitioner collects one or more tissue specimens. This may occur in the context of a variety of medical procedures. For example, the patient 10 may have tissue removed during an endoscopy procedure. As another example, the patient 10 may have a skin biopsy by a dermatologist. As yet another example, the patient 10 may have a tumor or organ completely removed by a surgeon. The specimens are placed in one or more bottles 14 having labels with RFID tags. Although described in terms of bottles, the system may use other containers to hold specimens, such as pots, boxes, or other suitable container. As described in further detail below, the RFID tags of the bottles may be programmed to include patient identification information, a bottle identifier (ID), a description of the stored sample, and other information. Alternatively, specimen management system 4 may update the patient record to record the unique identifiers for the RFID tags of the particular bottles used to contain the patient's specimens.

The bottles 14 are then transferred to a laboratory 16 (“lab 16”), such as an anatomic pathology laboratory, which may be at a different location within the institution or off-site. For example, the bottles 14 may be sent pneumatically via tubes from the examination or surgery rooms 12 to the laboratory 16. The RFID tags of the bottles may be interrogated at different locations during the process of transferring the bottles 14 from rooms 12 to laboratory 16. At laboratory 16, information may be read from the bottles 14 by an RFID reader associated with specimen management system 4. For example, the RFID reader may be used to check the bottles 14 into laboratory 16 by updating status information for the patient's record within SMS 4 to reflect that the bottles for the patient are now located in laboratory 16. SMS 4 may also be used to verify that a correct number of bottles 14 is present, i.e., that all of the bottles prepared by surgery 12 for a particular case have arrived and are collocated at laboratory 16 for processing. SMS 4 may also be used to verify that no mixing across patient cases has occurred, i.e., that the set of bottles belongs to only a single patient case. In addition, in some embodiments, information may be transferred between SMS 4 and a separate laboratory information system (LIS) database 22. LIS database 22 may be part of a laboratory information management system. SMS 4 may require that a complete set for the patient case has been presented before allowing information to be transferred from SMS 4 to LIS database 22. In other embodiments, network environment 2 may not include a separate LIS database 22.

At laboratory 16, the specimens contained within the bottles 14 are processed, as will be described in further detail below. At this time, tissue blocks 18 and slides 20 are typically prepared at laboratory 16, and each include an RFID tag. The term “block” may be used to refer to both the treated specimen (i.e., a dehydrated specimen embedded in wax), as well as the container that holds the specimen embedded in wax (which may also be referred to as a cassette). Unique identifiers for the RFID tags for blocks 18 and slides 20 are further recorded within the patient record within specimen management system 4, and the RFID tags may be programmed based on information stored within the RFID tag of the bottle 14 from which each block 18 and slide 20 originates. In this manner, specimen management system 4 helps ensure that the correct patient information is associated with each of the blocks 18 and slides 20. The slides 20 are then transferred to a pathologist office 24, while blocks 18 and any remaining bottles 14 may be transferred to archive 30. Alternatively, blocks 18 and bottles 14 may remain in laboratory 16 or be discarded.

Upon arrival at pathologist office 24, information may be read from slides 20 by another RFID reader associated with specimen management system 4. For example, the RFID reader may be used to check in the slides 20 to the pathologist office 24 by updating the patient record within SMS 4 to reflect that the slides 20 for the patient are now located at pathologist office 24. SMS 4 may also be used to verify that a correct number of slides 20 are present, i.e., that all of the slides 20 prepared for a given case have successfully arrived at the pathologist office 24. SMS 4 may also be used to verify that no mixing across patient cases has occurred, i.e., that the set of slides belongs to only a single patient case. The pathologist or an assistant may use the information stored on the RFID tags associated with slides 20 to access information about the associated patient using SMS 4 and/or LIS 22 or patient management system 9. The pathologist analyzes the specimens, such as by viewing slides 20 through a microscope, and produces a pathology report based on the analysis.

Once the pathologist office 24 is finished with slides 20, slides 20 may be sent to archive 30 for long-term storage. Upon arrival at archive 30, information may be read from the bottles 14, blocks 18, and slides 20 by another RFID reader within archive 30 associated with specimen management system 4. For example, the RFID reader may be used to check the bottles 14, blocks 18, and slides 20 into archive 30 by updating the patient record within SMS 4 to reflect that the bottles 14, blocks 18, and slides 20 are now located at archive 30. SMS 4 may also be used to verify that a correct number of bottles 14, blocks 18, and slides 20 are present for the particular case.

In this manner, RFID techniques are used to track patient-specific materials throughout a specimen collection and analysis process. The techniques may be used to ensure proper association between a patient 10 and the bottles 14, blocks 18, and slides 20, and ultimately with the pathologist report on the specimens. The techniques may be used to store digital information (e.g., unique RFID tag identifiers) within a specimen management system database to associate patient-specific materials with a patient record and/or to transfer digital information through a series of RFID tags through the process, i.e., from patient intake and initial collection of the specimens to ultimate reporting of results of the analysis to the patient.

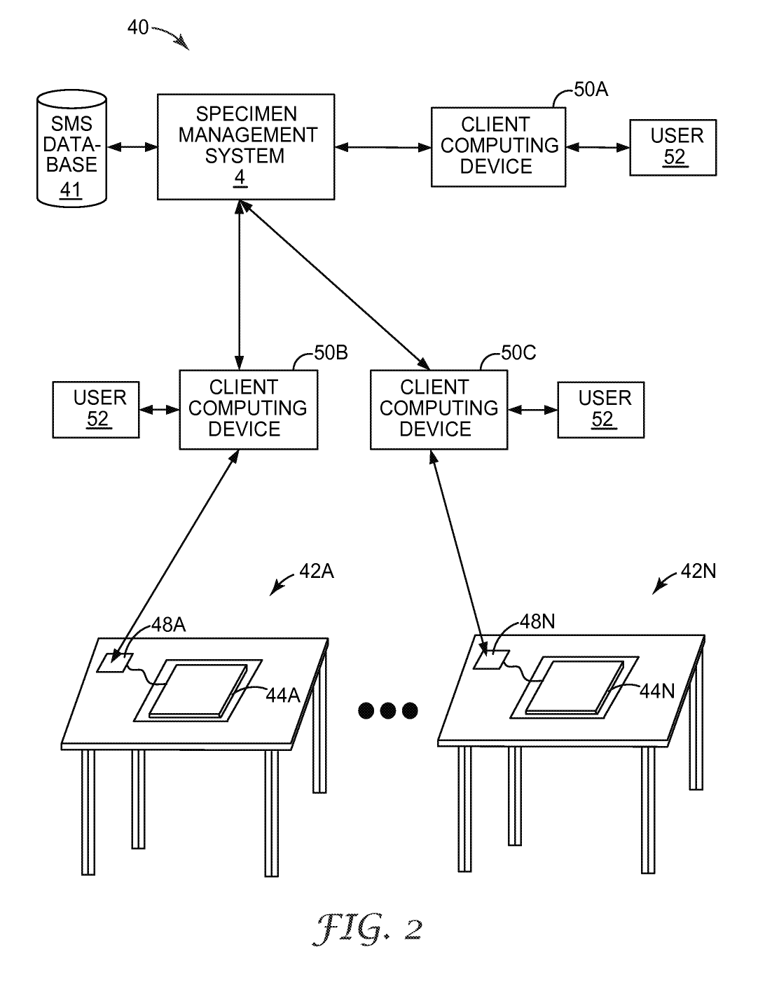

FIG. 2 is a block diagram illustrating a simplified view of certain components of network environment 2 within a healthcare facility. In this example, network environment 2 includes specimen management system (SMS) 4 that is used to track patients 10 and specimens held within or on physical containers, such as specimens within bottles 14, blocks 18, and slides 20 of FIG. 1. Additionally, clinic 8 may utilize SMS 4 to track other items, such as patient files, dictation about the collected specimen, pathology reports, or other items within the institution.

As described, network environment 2 utilizes RFID tags that uniquely identify the articles to which the RFID tags are affixed, e.g., by unique serial numbers associated with the RFID tags. In addition, SMS 4 may write additional information to the RFID tags, such as identification information for the particular patient or case with which a specimen is associated, information identifying a physical bottle or block from which the specimen originated, information identifying a user who has performed an action with respect to the specimen, one or more timestamps indicating a time at which the tissue was collected or processed, identification information for the surgery, laboratory, or pathologist, environmental information, and other information.

In general, RFID stations 42A-42N (“RFID stations 42”) of SMS 4 operate within a defined frequency range of the electromagnetic spectrum, such as 13.56 MHz with an allowable frequency variance of +/−7 kHz. However, other frequencies may be used for RFID applications. For example, some ultra-high frequency (UHF) RFID systems operate at approximately 900-928 MHz or 2-3 GHz. Different containers used within the process may have RFID tags operating within different frequency ranges. For example, bottles 14 may have UHF RFID tags, while blocks 18 and slides 20 have RFID tags that operate at 13.56 MHz. Other combinations are contemplated. In some embodiments, RFID stations 42 may be hybrid stations that utilize both RFID readers and barcode readers. Bottles, blocks, slides, or other articles may have barcodes in addition to or instead of RFID tags.

The RFID tags themselves may take any number of forms without departing from the scope of the present invention. Examples of commercially available RFID tags include 3M™ RFID tags available from 3M Company, Saint Paul, Minn., or “Tag-it” RFID transponders available from Texas Instruments, Dallas, Tex. An RFID tag typically includes an integrated circuit operatively connected to an antenna that receives RF energy from a source and backscatters RF energy in a manner well known in the art. The backscattered RF energy provides a signal that the RFID tag modulates to communicate information about the RFID tag and its associated article.

SMS 4 may provide or otherwise communicate with a database 41 to store the tag information for each bottle 14, block 18, slide 20, or other article used in the specimen collection and analysis process. Database 41 may be located within the healthcare facility. Alternatively, database 41 may be located at a remote location and remotely accessible via the healthcare facility. SMS 4 may be networked or otherwise coupled to one or more client computing devices 50A-50C so that users 52 at various RFID stations 42 can program the tags and/or access data relative to those items.

RFID stations 42 typically include RFID readers 48A-48N coupled to antennas 44A-44N (“antennas 44”) for interrogating the RFID tags fixed to articles and programming the RFID tags as desired. SMS 4 may also be coupled to a stand-alone client computing device not associated with one of RFID stations 42, such as client computing device 50A. A user 52 may use client computing device 50A to view the history of a particular specimen, e.g., where the specimen originated, its current and previous locations and timestamps at each location. Although not illustrated, SMS 4 may use other RFID readers, such as handheld RFID readers, doorway mounted or corridor RFID readers associated with examination and procedure rooms, intelligent storage locations having built-in RFID readers, and the like.

RFID stations 42 may be positioned at a variety of locations along the collection and processing process within the healthcare facility. For example, RFID stations 42 may be located within patient intake 8, examination or surgery rooms 12, laboratory 16, pathologist offices 24, or archive 30. For example, a user 52 at examination or surgery room 12 may print and encode labels having RFID tags for bottles 14 using an RFID printer/encoder (not shown) at RFID station 42A. User 52 may record collection of specimens and program the RFID tags on the bottles 14 by placing bottles 14 containing the specimens on or near an RFID station, e.g., RFID station 42A, and interacting with client computing device 50B. In particular, upon sensing and interrogating the RFID tags adhered to or embedded within the bottles 14, SMS 4 may record the unique identifiers for the RFID tags within database 41. SMS 4 may then access the RFID tags to program a date and time of collection. As further described below, SMS 4 may guide user 52 through a series of steps for verifying that all the necessary objects associated with the current case for a given patient 10 are present and properly programmed, and that objects from multiple patient cases are not detected. When items such as bottles 14, blocks 18, or slides 20 are in transit from one location to another, the RFID tags of each may be interrogated to check for completeness of the set, mixing of patient cases, to determine a destination for the bottles 14, blocks 18, or slides 20, or other verification procedures.

The RFID printer/encoder device used to print the labels may have a first RFID antenna having a limited read range and positioned proximate an output that produces the printed labels. The first RFID antenna may be used to program a label concurrently with printing the label. The RFID printer/encoder device may include an additional antenna that may be used for verifying that the RFID tags are properly programmed after they are removed from the printer and placed onto items. The additional RFID antenna may also be used for other tracking/programming tasks. The device would enable the user to control which antenna is used at a given time, and may employ a rapid switching method when neither antenna is specified.

Similarly, upon receiving a set of bottles 14 for a case at laboratory 16, a user 52 may use an RFID station 42 located within laboratory 16 to verify that all bottles 14 for the case are present and to record receipt of the bottles. In particular, upon sensing and interrogating the RFID tags embedded within the bottles 14, SMS 4 may record a timestamp within database 41 in association with the unique identifiers of the RFID tags. When blocks 18 are to be prepared out of a specimen from a bottle 14, user 52 may use RFID station 42 to program RFID tags on the blocks 18 using information stored on the RFID tag of the bottle 14, such as by simultaneously placing the bottle 14 and blocks 18 on antenna pad 44. Similarly, when slides 20 are to be prepared out of a specimen from a block 18, user 52 may use RFID station 42 to program RFID tags on the slides 20 using information stored on the RFID tag of the block 18, such as by simultaneously placing the block 18 and one or more of slides 20 on antenna pad 44. In this manner, network environment 2 ensures that the digital information is transferred from a bottle to the corresponding blocks, and from a block to the corresponding slides.

Users 52 within pathologist office 24 and archive 30 may similarly use RFID stations 42 to verify that all expected bottles 14, blocks 18, and slides 20 for the case are present, and to record receipt of the bottles at the given location within database 41 of SMS 4. In this manner, SMS 4 may be used with RFID stations 42 at various points in the specimen collection and analysis process to establish and maintain a chain of custody of the specimens, and to ensure that the proper patient identity and case information is associated with each bottle 14, block 18, and slide 20 during preparation and analysis.

In some embodiments, SMS 4 may in part provide an RFID inventory management system to manage inventory of incoming bottles, blocks, slides, and other articles. The healthcare facility may include “smart” storage areas (e.g., shelves, vertical files, carts, bins, cabinets, boxes or other locations) that are RFID-enabled. The smart storage areas may be equipped with one or more antennas for interrogating RFID tags to aid in determining in real-time which articles are located at each of the storage areas. The antennas may be positioned in various ways, such as on top or bottom of a storage area, at the back of a storage area, or supported vertically, interspersed among the articles. The antennas can be retrofitted to existing storage areas or built into a storage area and purchased as a unit.

The information collected by SMS 4 may be useful in locating specimens as well as tracking, for example, cycle time and efficiency of one or more people who work with the specimens. One example of an RFID tracking system incorporating “smart” storage areas and an exemplary RFID antenna for use in such a system is described in co-pending and commonly assigned U.S. Pat. No. 6,861,993 entitled “MULTI-LOOP ANTENNA FOR RADIO-FREQUENCY IDENTIFICATION,” filed Mar. 3, 2003 to Michele A Waldner, hereby incorporated by reference.

SMS 4 typically includes a plurality of software modules executing on one or more servers, e.g., web servers, application servers and/or database servers, to perform the functions described herein. The software modules include instructions executable by a programmable processor and may be stored in a computer-readable storage medium, such as memory or a disk. SMS database 41 may be deployed across one or more database servers, and may be a relational database, multidimensional database, object-oriented database, associative database, a series of flat files or other suitable data storage mechanism.

In one embodiment, SMS 4 includes software to present an administrative user interface to allow an administrator or other authorized user to configure the SMS. By interaction with the user interface, the administrator may define a set of rules. Each rule defines one or more criteria and an associated action to be performed by SMS 4 upon triggering of the respective rule, i.e., satisfaction of the rule's criteria. SMS 4 stores the defined rules within SMS database 41.

A rules engine within SMS 4 evaluates the rules in view of data within SMS database 41, patient management system 9, and laboratory information system 22, such as data related to patients, containers, specimens, time periods and the like. SMS 4 may, for example, evaluate the rules periodically or in response to an event, such as an expiration of a timer or receipt of new data from any of client computing devices 50 in response to interrogation of an RFID tag.

As one example, the administrator may configure a set of one or more rules to define a route for each type of anatomical pathology specimen container used within the health care facility. That is, the route may specify two or more different locations within the facility at which the container is expected to be interrogated (i.e., sensed or programmed). For example, the administrator may configure one or more rules to specify an expected route for bottles 14 as surgery 12→laboratory 16→archive 30. As another example, the administrator may configure one or more rules to specify an expected route for slides 20 as laboratory 16→pathologist 24→archive 30. In this manner, the defined route specifies the sequence of locations a particular type of container is expected to traverse after creation, i.e., upon being programmed and associated with a patient record.

The set of rules may also define a maximum expected time period for the type of anatomical pathology specimen container to travel between two of the locations on the route or along the entire route. SMS 4 interrogates the RFID tag of the anatomical pathology specimen container with RFID readers at each location upon receiving the anatomical pathology specimen container at the respective location or shipping the container from the location. SMS 4 stores a timestamp to indicate the time and location that the anatomical pathology specimen container was received and/or shipped. SMS 4 may start a timer when an RFID tag for a given container is interrogated at a particular location, such as recording that the RFID tag and the corresponding container has been checked-in or out at the location. Failure to arrive at the next location (i.e., the “destination” location) along the route within the defined expected time period after having been checked-out at the previous location (i.e., the “source” location) provides an indication to SMS 4 that the container may be lost.

SMS 4 may provide a variety of alerts to users based on firing of one or more rules. The alerts may be delivered as messages on client computing devices 50, via email, page, automated voice message, or other means. SMS 4 may, for example, provide an alert upon determining, based on the interrogation of an RFIG tag at one or more of the locations along the route, that shipment of an anatomical pathology specimen container within the medical facility fails to comply with the route defined for that type of container. As another example, SMS 4 may automatically provide an alert when the anatomical pathology specimen container skips an expected location along the route or is detected at the same location more than once. As another example, SMS 4 may automatically provide an alert when the anatomical pathology specimen container exceeds any expected time period, such as the maximum expected time period for the type of anatomical pathology specimen container to travel between any two of the locations along the route or to traverse the entire route.

The administrator may also interact with SMS 4 to specify one or more rules defining an expected number of anatomical pathology specimen containers to be associated with a patient record for different types of medical procedures. For example, the administrator or clinician may create a rule specifying that a skin biopsy is expected to produce one bottle 14. As another example, the administrator or clinician may create a rule specifying that a procedure for removal of all or a portion of a tumor is expected to produce one to four bottles 14.

The administrator may also interact with SMS 4 to specify one or more rules defining an expected number of anatomical pathology specimen containers to be used to store portions of specimens previously held by containers of a different type. For example, the administrator may create a rule specifying that one bottle 14 of a specimen is expected to produce at least one and no more than ten tissue blocks 18.

The administrator may also define rules specifying a time period between association of an anatomical pathology specimen container of one type (e.g., a bottle or block) with a patient record within SMS 4 and association of an anatomical pathology specimen container of a second type (e.g., a block or slide) with the patient record. In this manner, a rule may be created specifying, for example, that association of a tissue bottle with a patient record should be followed by association of at least one tissue block with that same patient record within the defined time period, e.g., twenty-four hours. SMS 4 monitors data received from client computing devices 50, starts internal timers upon detecting the association of tissue containers with a patient record, and generates alerts in the event the number of containers associated with a patient record fails to conform to the expected number or in the event containers of certain types are not associated with the patient record within the specified expected time periods.

In addition, the administrator may interact with SMS 4 to specify one or more rules defining an expected sequence of actions to be performed with respect to a patient or particular type of tissue specimen. For example, a rule may be created to specify that a certain type of tissue is expected to be collected at a particular surgical location 12. The rule may further specify that this type of tissue sample is expected to be sent to laboratory 16 for one or more of a defined list of procedures or tests (referred to generally as actions). The particular locations and list of expected actions for each location may vary depending on the type of specimen being managed by SMS 4. Similar rules may be defined based on type of medical condition. Based on data collected by interrogation of the RFID tags for the specimen containers, as well as data received from patient-specific data received from patient management system 9 or laboratory information system 22, SMS evaluates the rules and provides alerts upon determining that one or more actions within the expected sequence of actions has failed to occur.

As another example, the administrator may interact with SMS 4 to specify one or more rules that are triggered based on interrogation of the RFID tag associated with a patient identification bracelet or other identification article. For example, the administrator may define rules to provide an alert when an examination room or laboratory 12 at which the patient is detected does not correspond to the examination room or laboratory to which the patient was assigned. The patient identification bracelet may be interrogated at the examination room or laboratory 12 using a handheld RFID device or using a doorway RFID reader as the patient enters the examination room.

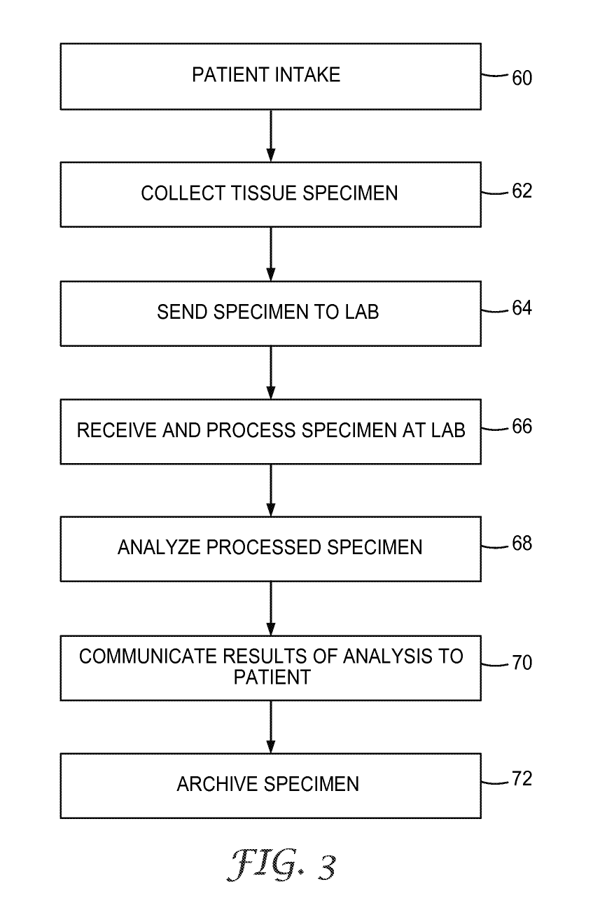

FIG. 3 is a flowchart illustrating an exemplary process performed by a healthcare facility in utilizing the RFID specimen management and tracking techniques described herein. FIG. 3 provides a basic overview of a process of collecting, processing, and analyzing one or more specimens obtained from a patient. The individual steps of the process will then be described in further detail below.

Initially, upon a patient's arrival at the healthcare facility, patient identity and other information are obtained from a patient during patient intake 8 (60). The healthcare facility may create or update a patient record within patient management system 9 to reflect that the patient has checked in. A patient record may also be created or updated within SMS database 41 during patient intake; alternatively, this may occur at a later time, such as when a specimen is collected from the patient in an examination or surgery.

During surgery or examination, tissue specimens are collected from the patient 10 by a surgeon or other medical practitioner (62). The specimens may be placed in containers, such as bottles 14, that are labeled with RFID tags, and specimen management system 4 is updated to associate the RFID tags with the patient record. The bottles 14 are sent to an anatomic pathology laboratory (64). The laboratory receives the bottles 14, processes the specimens from bottles 14 into blocks 18, and processes the specimens from blocks 18 into slides 20 (66). The specimen from which other specimens are cut or otherwise obtained may be referred to as a source specimen. For example, the specimen in a bottle is the source specimen for each of the specimens processed into blocks from that specimen. Specimen management system 4 is updated to record the RFID tags of the blocks 18 and slides 20 for the patient record, and the slides 20 are sent to a pathologist, who analyzes the specimens and produces a pathology report (68). A medical practitioner, such as the surgeon who performed the specimen collection, may communicate the results of the pathologist's analysis to the patient (70). Remaining bottles 14, blocks 18, and slides 20 may then be archived, an action which is reflected in the information maintained by specimen management system 4 (72).

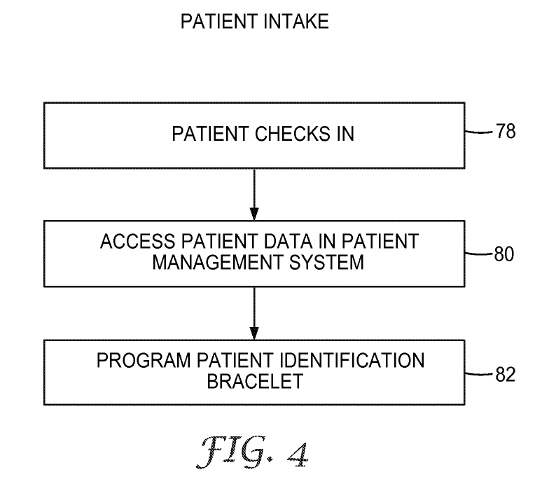

FIG. 4 is a flowchart illustrating in further detail an example process in which RFID tags are utilized during patient intake. A patient checks in to the healthcare facility by providing patient identity and other information (78). If data relating to the patient already exists within the patient management system 9, the data may be accessed (80), and updated to reflect that the patient has checked in. If the patient is not already represented in patient management system 9, the patient's information may be added to patient management system 9 at the time of check in. The patient's personal information may be associated with a unique patient identifier, i.e., record number, within patient management system 9. At this time, a patient record may also be created in SMS 4, and a patient identification article containing an RFID tag may be provided to the patient.

The patient identification article may take a variety of forms, such as a bracelet, ID badge to be worn around the neck or pinned to clothing, an adhesive-backed ID tag attached to clothing, or other articles. The RFID tag of the patient identification may be programmed with patient identification information, such as the unique patient identifier of patient management system 9 (82) and provided to the patient to be worn during the patient's stay at the healthcare facility. In addition, the patient record within SMS 4 may be updated to record a unique serial number within the RFID tag of the article (e.g., bracelet) as well as the patient identification information from patient management system 9. The patient identification bracelet may also include a human-readable label and/or machine-readable information, e.g., a bar code.

The patient may be associated with a procedure room during the check-in process, and this association may be stored within an information management system such as patient management system or SMS 4. When the patient is received at the procedure room, the RFID tag of the patient identification article may be interrogated, and the information management system accessed using the patient identification information obtained from the RFID tag. A doorway RFID reader, handheld RFID reader, or room-wide RFID reader may be used to interrogate the RFID tag associated with the patient. The information management system may be used to verify that the patient is at the correct surgical location, e.g., by determining whether the patient is in the procedure room with which the patient was associated. The information management system may provide an alert if a patient is detected in an incorrect surgical location.

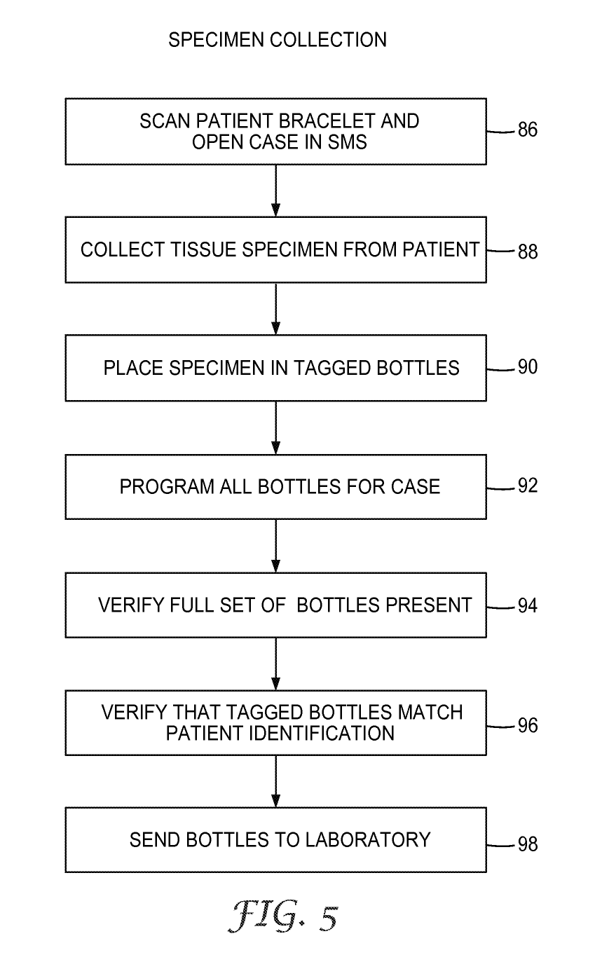

FIG. 5 is a flowchart illustrating in further detail an example process in which RFID tags and specimen management system 4 are used during specimen collection. When a patient enters an examination or surgery room, an assistant may scan the patient's identification bracelet with an RFID reader and, if not already created, opens a new case for the patient in SMS 4 (86). For example, SMS 4 and patient management system 9 may be linked such that when SMS 4 obtains the patient's unique patient identifier from the RFID tag on the patient identification bracelet, SMS 4 can import patient data from patient management system 9 to SMS 4. Alternatively, SMS 4 may simply store a pointer or reference to the appropriate patient data in patient management system 9 without importing all of the patient data. As another example, the assistant may manually enter patient data into SMS 4 via a user interface. In one example embodiment, the user interface may be a touch screen interface. In any case, the assistant opens a new case entry in SMS 4, which may have a unique case identifier. The new case within SMS 4 will store all data relating to the specimens to be collected from the patient on this occasion.

A medical practitioner then proceeds to collect a tissue specimen from the patient (88). As described above, any of a variety of procedures for specimen collection may be employed depending on the particular patient's circumstances. For example, tissue from the esophagus or colon may be collected by way of an endoscopy. The medical practitioner or an assistant places the specimen in one or more tagged bottles (90). The bottles may have a human-readable label and/or machine-readable label as well as an RFID tag. The RFID tag may be integrated as part of the human-readable label, or the two may be separately affixed to a bottle. As one example, an RFID tag may be built into the side or lid of the bottle so as not to obscure the view of the bottle contents. As another example, a substantial portion of the RFID tag may be transparent. In one embodiment, bottles may be provided by a bottle dispenser that includes a printer/encoder that prints the human-readable label and encodes (programs) the RFID tag on the label with a unique identifier as bottles exit the unit. The label may be adhered to the bottle before, during, or after the bottle exits the unit. Each item may be programmed with a globally unique ID. The RFID-enabled bottle dispenser may be configured to dispense bottles in a sterile manner. An assistant may actuate a button or click a mouse to automatically initiate printing, programming, and dispensing of the bottle. Alternatively, a bottle dispenser may simply dispense sterile bottles, and a label and/or RFID tag may be printed/programmed after tissue is placed within the bottle.

The bottles may be programmed with information, such as information obtained from the patient identification bracelet or SMS 4, or information describing the specimens that is manually entered by the assistant (92). Programming of the bottles may be initiated automatically by SMS 4, or may be initiated by a user such as the assistant. In one embodiment, the bottle dispenser may include an internal RFID reader/programmer to program the RFID tag of a new bottle with the appropriate case identifier, patient identifier, procedure identifier, or other information, and to record a timestamp and location in SMS 4 to identify when and where the new bottle was first used.

In another embodiment, an assistant may program the bottles at a separate RFID station 42 (FIG. 2) having an RFID antenna 44 using a client computing device 50. The client computing device 50 may present a user interface for accessing SMS 4, and the user interface may guide the assistant through the process of programming each of the bottles. For example, the software application may allow the assistant to verify that the full set of bottles is present and has been successfully programmed (94). The software application may also prompt the user to verify that the tagged bottles match the patient identification stored on the patient identification wristband or patient management system 9, which may be available by way of an application programming interface (API) (96). At the beginning of the programming process, a user may select the appropriate case information from a list of cases presented by SMS 4. The list may include only “likely” cases that have passed through filters applied by SMS 4, such as selection filters for selecting cases based on recentness of the procedure, procedure location, type of procedure and the like. For example, a healthcare facility may use a central programming station rather than programming the bottles in the surgical room. After surgery, a user brings the bottles with the specimens out to the central programming station, select their case, and program the bottles based on the selected information.

When programming the RFID tags of the bottles, the user may select one of a plurality of pre-programmed RFID tags pre-programmed with procedure IDs associated with different types of surgical procedures. The user may then present the selected RFID tag pre-programmed with a procedure ID to RFID antenna 44 to easily and quickly program the bottles and/or update SMS 4 with information about the particular type of procedure through which the specimen was obtained. In one embodiment, RFID antenna 44 may be coupled to a telephone system (e.g., via client computing device 50), and presenting the procedure ID of the pre-programmed RFID tag to RFID antenna 44 may cause SMS 4 to control the telephonic equipment to dial a dictation phone number. SMS 4 may then automatically upload and record some standard dictation in response to the pre-programmed RFID tag. In addition, the medical practitioner who performed the procedure may then be prompted to enter his or her specific dictation for the case.

In this manner, SMS 4 ensures that the bottles are properly labeled and associated with the correct patient information. The bottles may then be sent to an anatomical pathology laboratory for processing of the specimens (98). Checks may be performed on the bottled during transit to the laboratory. The laboratory may be at a central location within the healthcare campus with respect to the office at which the specimen is collected, or may be at a location off-site. Where the laboratory is within the healthcare campus, the bottles may be transferred to the laboratory via a pneumatic transfer mechanism or other method of transfer. Information about where the bottles should be transferred may be stored on the RFID tags on the bottles or associated with the patient record within SMS 4. For example, a user may place the bottles within a pneumatic device having an embedded RFID reader that reads the RFID tags of the bottles to determine the correct destination(s) for the bottles, and pneumatically sends the bottles to the destination(s) defined by the RFID tags. The initial programming of the bottles may automatically trigger alerts (e.g., electronic messages, events, emails) to other departments, such as the anatomical pathology laboratory, that specimens of a given type and number were collected and should soon be expected to arrive at the laboratory. This may allow the laboratory to plan ahead for incoming specimens, and may assist in preventing lost bottles from going unnoticed. In addition, SMS 4 may cause some samples to be automatically redirected to alternative laboratories based on laboratory capacities and the quantity of specimens entering the system. In such case, SMS 4 may interact with LIS 22 by way of an API to inform the laboratory of any change to the destination.

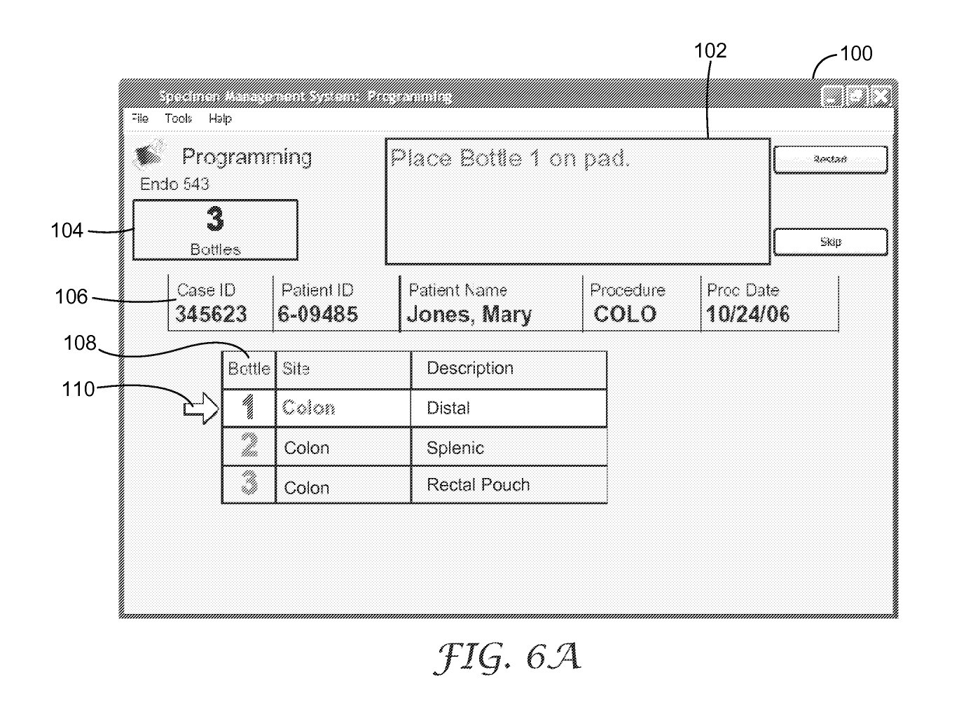

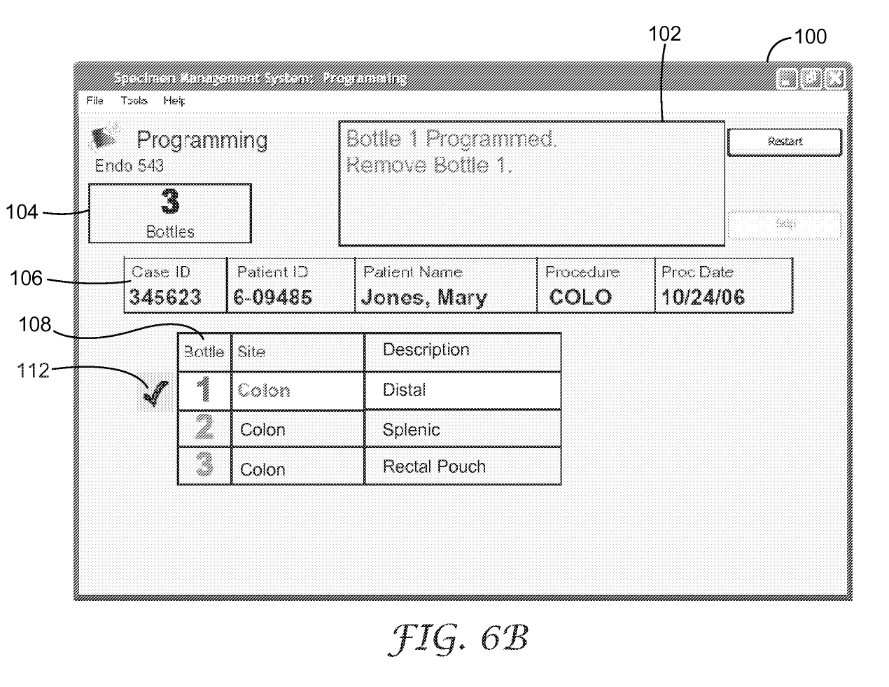

FIG. 6A is a screen illustration illustrating an example user interface 100 presented by an RFID station (e.g., RFID station 42A of FIG. 2) of specimen management system (SMS) 4 for programming specimen bottles. A user may interact with SMS 4 via user interface 100, which may be presented on a computing device such as client computing device 50A (FIG. 2). As shown in FIG. 6A, user interface 100 includes a message window 102 that presents prompts or feedback messages to the user. In the example shown, message window 102 instructs the user to place Bottle 1 on the pad (e.g., antenna 44A). User interface 100 also includes a total bottle count field 104 that indicates the total number of bottles for the present case, and a case entry line 106 that provides information about the present case. For example, case entry line 106 includes the case identifier (ID), the patient ID, Patient Name, Procedure Identifier, and Procedure Date. User interface 100 further includes a bottle table 108 that indicates information relating to the bottles to be programmed. An arrow 110 highlights the information relating to the current bottle to be programmed, i.e., Bottle 1. Bottle table 108 may include information such as the bottle number, the site from which the specimen originates, and a description of the specimen. In response to reading the prompt in message window 102, the user places Bottle 1 onto antenna pad 44A.

FIG. 6B is a screen illustration illustrating another example user interface 100 of SMS 4 for programming specimen bottles. Message window 102 of FIG. 6B shows that Bottle 1 has been programmed, and instructs the user to remove Bottle 1 from antenna pad 44A. A check mark 112 indicates that Bottle 1 has been successfully programmed. SMS 4 may similarly prompt the user to place Bottles 2 and 3 on the pad to be programmed in order until all of the bottles for this case have been programmed.

In some embodiments, SMS 4 may use a walk-away timer such that specific case information will be removed from user interface 100 after a time-out period of no activity. This ensures that displayed information is not kept visible when items are left unattended on an RFID tracking pad, and may help protect patient information privacy.

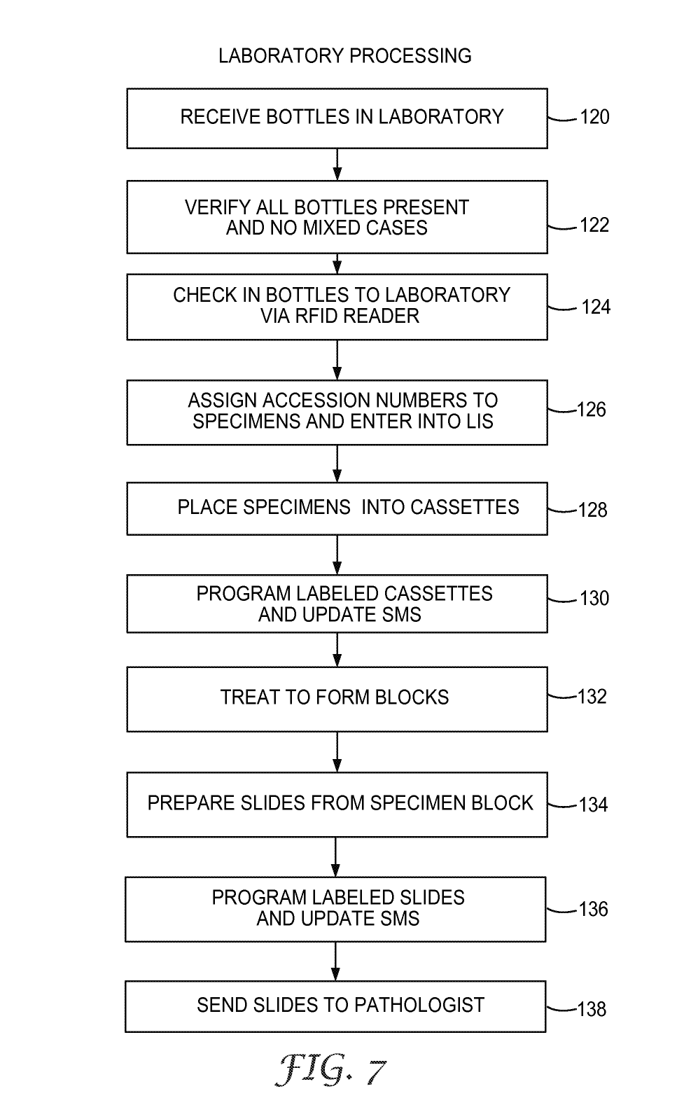

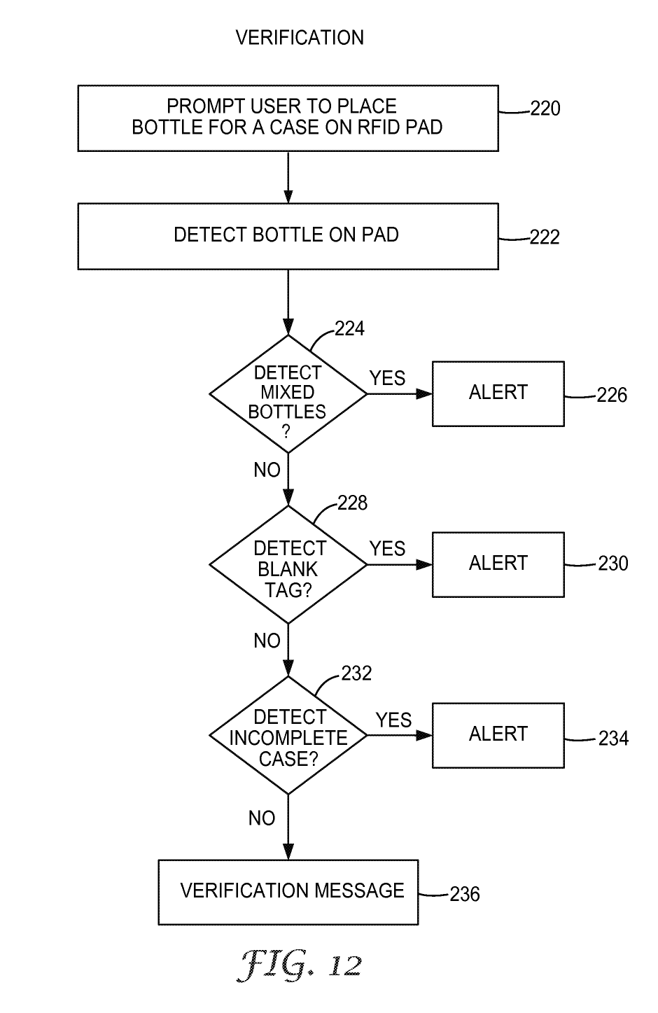

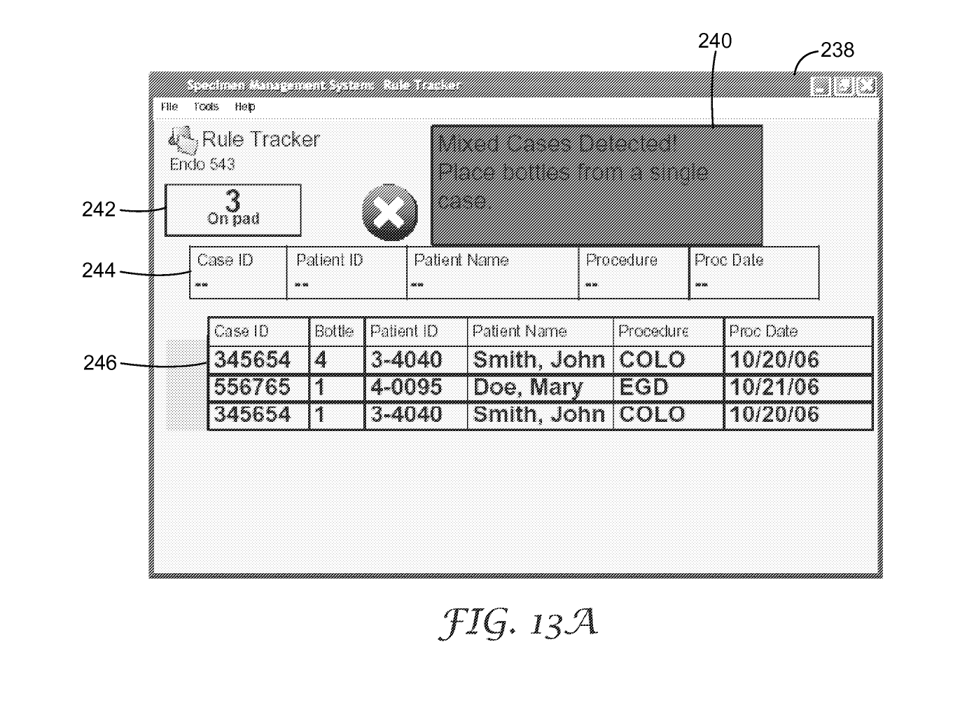

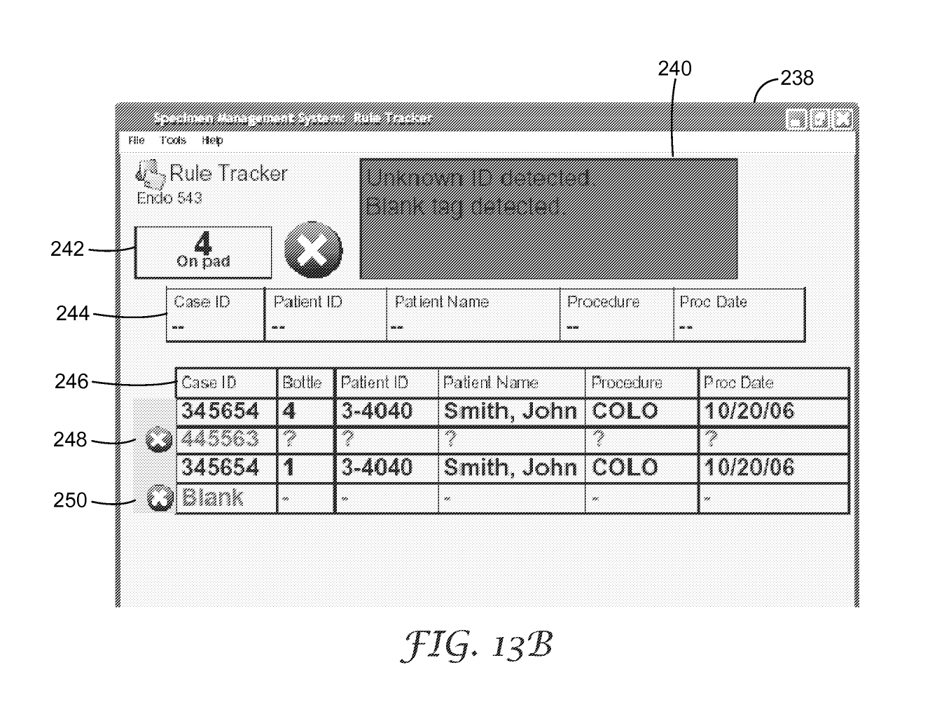

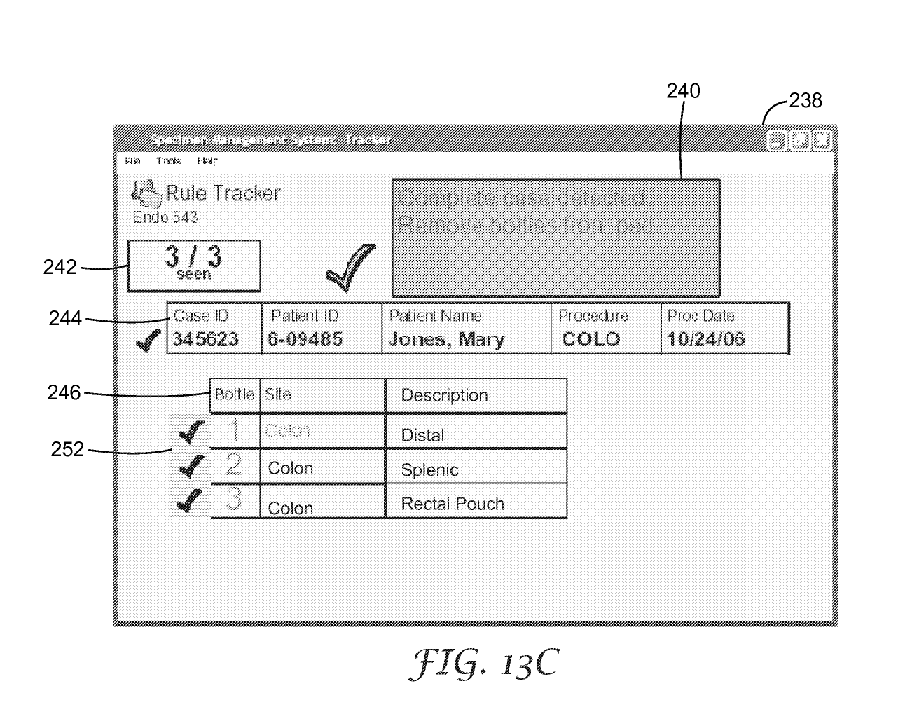

FIG. 7 is a flowchart illustrating in further detail an example process in which RFID tags and SMS 4 are used during laboratory processing. Bottles for a given case are received in laboratory 16 (120), and SMS 4 may prompt a user via a user interface to present the bottles within a range of an RFID reader for RFID interrogation to verify that all bottles for the case are present and no incorrect (mixed) bottles are within the set (122). The bottles may have a specific order associated with them, and SMS 4 may prompt the user to present the bottles sequentially in a predefined order, and may provide an alert to the user when the RFID reader reads the RFID tags of the bottles in an order other than the predefined order. The verification may be performed at an RFID station 42 located within the laboratory. An example verification process is described in detail below with respect to FIGS. 12 and 13A-13C. At this time, the user may also check in the bottles to laboratory 16 via an RFID reader 48 at the RFID station 42 (124). Checking in the bottles may cause SMS 4 to create a new entry in SMS database 41 to record a timestamp that indicates the bottles were verified as present in laboratory 16 at the time indicated by the timestamp.

The bottles may also be assigned accession numbers that are entered into a separate laboratory information system (LIS) database 22 (126) (FIG. 1). The accession numbers may also be recorded by SMS 4. Alternatively, accession numbers may be automatically populated in LIS database 22 based on information read from the RFID tags on the bottles, such as the case ID and bottle number, or may be directly transferred from SMS database 41 to LIS database 22. In either case, the information stored in LIS database 22 would be consistent with the information stored in SMS database 41. In one embodiment, laboratory 16 does not include a separate LIS database 22, but simply references SMS database 41. In some embodiments, SMS 4 may include a data dictionary to convert terminology used in surgery to terminology used in the LIS. This enables practitioners in each area to continue using their own established terminology without resulting in inconsistency.

The specimens from the bottles may then be processed into blocks (128). For example, a specimen from a bottle may be cut into smaller pieces, and the pieces may each be placed within separate cassettes. The cassettes may include a label and RFID tag already (e.g., the cassettes may be manufactured to include the label and RFID tag), or a label and RFID tag may applied to the cassettes when the specimens are placed in the cassettes. The label and the RFID tag may be printed and encoded at laboratory 16, similar to the process described above with respect to bottles in the surgery room. Although described below in terms of programming RFID tags of the cassettes prior to placing the specimens on the cassettes, in some embodiments the RFID tags may be programmed after the cassettes and specimens have been treated to form blocks.

The unique serial numbers for the RFID tags for the cassettes or blocks are recorded within SMS 4 and associated with the patient record. Other information may be stored in SMS 4 as well and associated with the patient record, such as a block number, and the number of the bottle from which the specimen originated. In addition, the RFID tags on each block may be programmed with similar information including the case identifier, patient identifier, a block number, and the number of the bottle from which the specimen originated (130).

As will be described in further detail below, cassettes or blocks may be programmed directly from the bottle from which the specimens on the blocks originated, such as by placing the bottle on an RFID antenna pad 44 at an RFID station 42, and sequentially placing the cassettes or blocks on the RFID antenna pad 44. SMS 4 may prompt the user to place first one block on the RFID antenna pad 44 with the bottle, and upon programming the RFID tag of the block, prompt the user to remove the first block. SMS 4 may then prompt the user to place a second block on the RFID antenna pad 44 with the bottle, and the RFID tag of the second block is then programmed, and so on until all cassettes or blocks for the case have been programmed. Upon detection the presence of the RFID tag associated with the source bottle and the RFID tags for a block, SMS 4 may transfer or copy information from the RFID tag of the bottle to the RFID tag of the block. Alternatively, detection of the tags may be a pre-requisite to triggering recordation of the unique serial numbers of the RFID tag for the block within the patient record of SMS 4. In either case, this ensures that the correct patient information and bottle information is associated with each block produced by the laboratory. The specimens in the cassettes may then be treated to form specimen blocks, such as by dehydrating the tissue, adding paraffin or wax, and cooling the blocks (132). RFID tags of cassettes may be programmed before receiving a specimen, after receiving the specimen but before being processed into blocks, or after being processed into blocks.

Slides of the specimens may also be prepared at the anatomical pathology laboratory (134). For example, a specimen from a block may be finely shaved into smaller pieces using a microtome, with the pieces being placed in a water bath before being placed on individual slides. The slides may be manufactured to include the label and RFID tag, or a label and RFID tag may applied to the slides when the specimens are placed on the slides. An RFID tag may be designed to extend around the label space, or to extend around the perimeter of the slide or folded into a three-dimensional antenna to allow for a longer read range. The label and the RFID tag may be printed and encoded at laboratory 16, similar to the process described above with respect to bottles in the surgery room. The slides may then be stained, and slip covers added. The slides may be placed into books that hold many slides, and the books may also include RFID tags.

SMS 4 is updated to record the serial numbers of the RFID tags of the slides and associate the serial numbers with the current patient record (136) SMS 4 may also record for each slide a block identifier and/or bottle identifier from which the tissue on the slide originated. The RFID tags on each slide may also be programmed with information such as the case identifier, patient identifier, a block number, and the number of the bottle or block from which the specimen originated. As will be described in further detail below, slides may be programmed directly from the block from which the specimens on the slides originated, and the user interface of SMS 4 may require that the block and one of corresponding slides be placed together on an RFID antenna pad 44 at an RFID station 42 before recording any information and providing a verification indicator to the user. In other words, SMS 4 may permit programming the RFID tag of the block or slide only when the RFID tag of the block or slide and the RFID tag of the corresponding bottle or block are both within the range of the RFID antenna at the same time. Similarly, SMS 4 may require that the RFID tag of the block and the RFID tag of the corresponding bottle are both detected within a pre-defined time period before permitting programming of the RFID tag of the block. This ensures that the correct patient information is propagated along from the block to each individual slide.

As another example, a microtome for slicing specimens into sections for slides may include an embedded RFID reader to automatically determine which block is being sliced; this information may then be used for automatically programming the slides. A water bath may also include an RFID reader to automatically program slides to correspond to the block in the microtome. For example, an RFID reader may be placed near the water bath, and slides may be programmed by placing the slides near the RFID reader as the specimens are moved out of the water bath and onto the slides. As a quality control measure, the RFID readers may be coordinated to only allow programming of slides when the associated block and/or bottle is present at the same time. As yet another example, the surface used for placing specimens onto slides may include an RFID reader. In one example embodiment, data contained on the slide RFID tag may automatically drive a staining machine to stain the slide in a particular manner. As another example, data on the slide RFID tag may alert the user to the proper staining regimen for the specimen. The slides may then be sent to a pathologist office 24 for analysis (138).

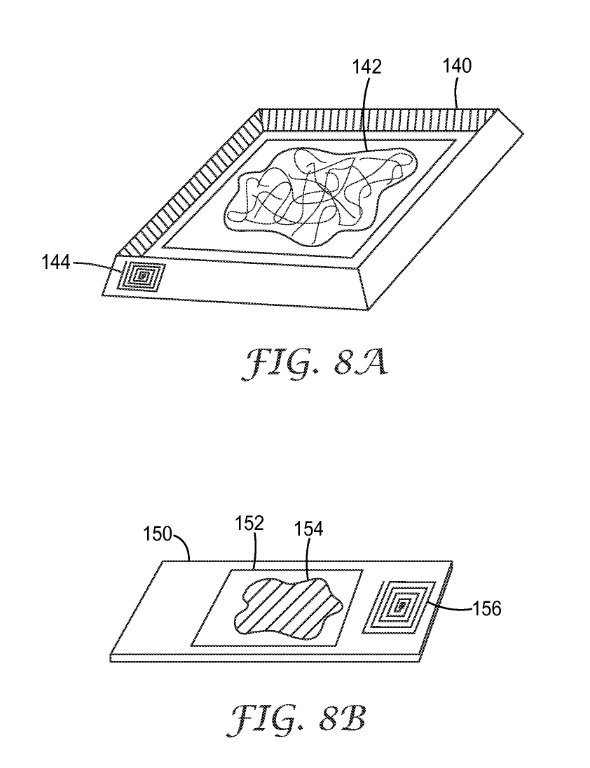

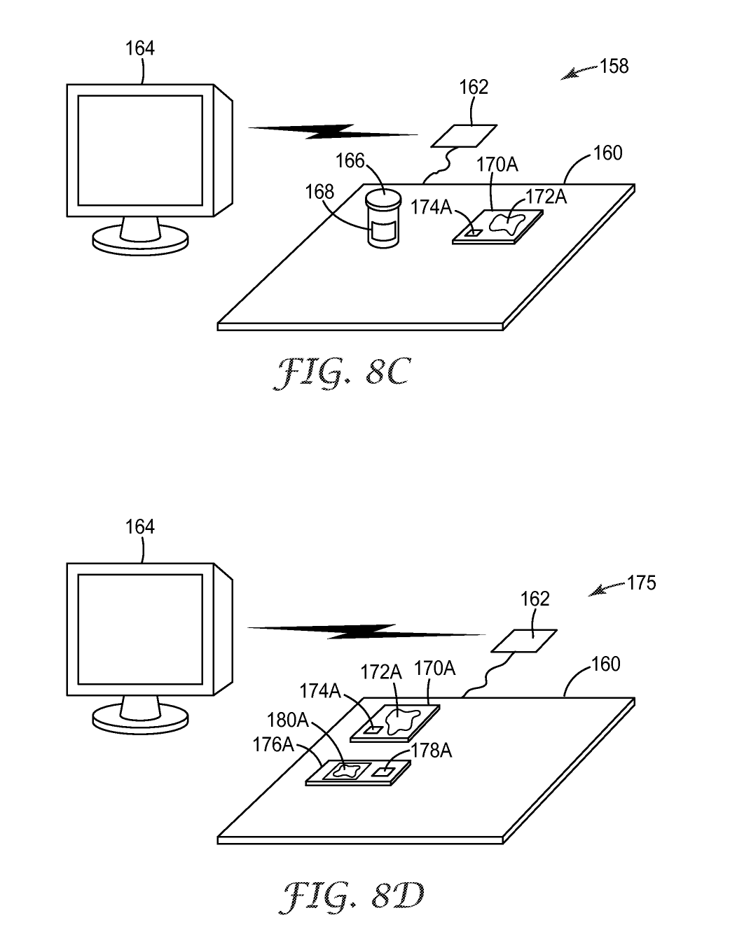

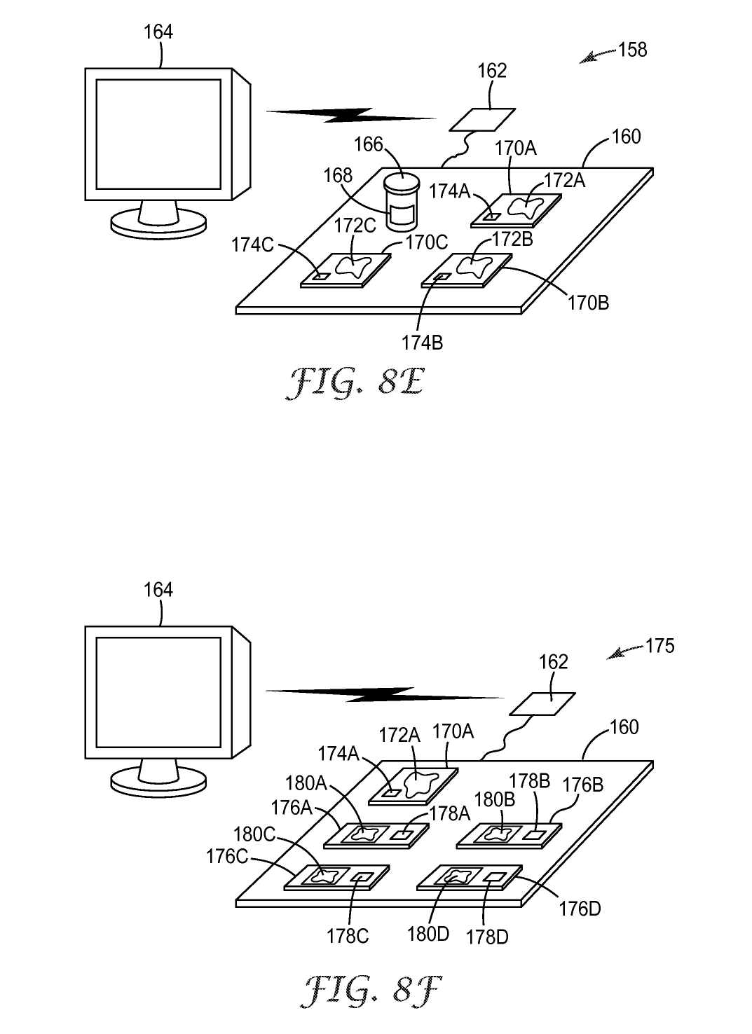

FIG. 8A is a block diagram illustrating an example specimen block 140 having an RFID tag 144 affixed thereto. Block 140 holds a tissue specimen 142. Specimen 142 may be a portion of a specimen received in a bottle. As described above, RFID tag 144 may be programmed at an anatomical pathology lab before or after specimen 142 is placed within specimen block 140, and may be programmed with information obtained from or associated with the RFID tag of the bottle from which specimen 142 originated.

Specimen block 140 may also include a human-readable and/or machine-readable label (not shown) including information that identifies the specimen, case number, or patient. RFID tag 144 may be built into specimen block 140 during manufacture of specimen block 140 (i.e., when specimen block 140 is a cassette), or may be affixed to specimen block 140 at the healthcare facility when specimen block 140 is initially to be used. Because specimen block 140 may be exposed to liquids or chemicals and undergo temperature extremes during dehydration, RFID tag 144 may be specially constructed to withstand these environmental conditions.

FIG. 8B is a block diagram illustrating an example specimen slide 150 having an RFID tag 156 affixed thereto. Slide 150 holds a specimen 154 under a slip cover 152. Specimen 154 may be a portion of a specimen from a block. As described above, RFID tag 156 may be programmed at the anatomical pathology lab when specimen 154 is placed onto slide 150, and may be programmed with information obtained from or associated with the RFID tag of the slide from which specimen 154 originated.

Slide 150 may also include a human-readable and/or machine-readable label (not shown) including information that identifies the specimen, case number, or patient. RFID tag 156 may be built into slide 150 during manufacture of slide 150, or may be affixed to slide 150 at the healthcare facility when slide 150 is initially to be used. Because slide 150 may be exposed to liquids or chemicals and undergo temperature extremes during dehydration, RFID tag 156 may be specially made to withstand these environmental conditions. A printer may be used in laboratory 16 for printing onto blocks or slides. The human-readable label and the RFID tag may be printed/encoded based on information manually entered by a user, or based on information obtained from SMS database 41. An RFID reader/writer may be included with the printer to program the RFID tag as the human-readable label for the block or slide is being printed. This may be advantageous since it combines printing and programming in a single step, and may ensure that the printed information matches the programmed information.

FIG. 8C is a block diagram illustrating an example RFID station 158 for programming RFID tags. RFID station 158 includes an RFID antenna pad 160, an RFID reader 162, and a client computing device 164 that presents a user interface to guide a user through the process of programming items having RFID tags. The user may interact with RFID station 158 for programming RFID tag 174A on specimen block 170 using an RFID tag 168 on specimen bottle 166. SMS 4 may provide a software application program accessible to the user via the user interface of client computing device 164. SMS 4 (via the software application program) requires the user to place certain items on RFID antenna pad 160 to be programmed as a prerequisite to programming RFID tag 174A of block 170A.

For example, SMS 4 may require that the user place bottle 166 as well as block 170A having a specimen 172A that came from bottle 166 on the pad 160 prior to transferring patient information from the RFID tag of the bottle to the RFID tag of the block. SMS 4 may permit programming of the RFID tag of the block only when both the RFID tags are detected within a time period of each other. RFID reader 162 may read information from RFID tag 168 of bottle 166, and write this information to RFID tag 174A of block 170A or update SMS 4 to associate block 170A with bottle 166. For example, the information may include the case ID associated with specimen 172A on block 170A. Upon programming RFID tag 174A of block 170A, SMS 4 may prompt the user to remove block 170A from RFID antenna pad 160. Upon detecting that the user has removed block 170A, SMS 4 may prompt the user to place a second block on RFID antenna pad 160. SMS 4 may prompt the user to sequentially place and remove blocks on RFID antenna pad 160 in a predefined order by referring to an identifier of the blocks (i.e., “Place Block 1 on pad”). In this manner, SMS 4 guides the user to program a set of blocks 170 for a patient case with information matching the corresponding bottle 166 from which specimens 172 originated. As a result, it is known that blocks 170 are related to bottle 166. For example, RFID tags 174 of blocks 170 may be given unique IDs that are based on the ID of bottle 166. SMS 4 may also interrogate RFID tag 174A after programming RFID tag 174A to verify that the information programmed to the RFID tag 174A is correct based on information read from RFID tag 168 of bottle 166.

SMS 4 may detect that an incomplete set of items has been presented (e.g., within a time period), and provide an alert to the user. SMS 4 may allow the user to override the alert and proceed with processing the items, and may also record in SMS database 41 an indication of that the user overrode the alert.

FIG. 8D is a block diagram illustrating an example RFID station 175 for programming RFID tags. RFID station 175 may include the same RFID antenna pad 160, RFID reader 162, and client computing device 164 as described with respect FIG. 8C, which may also be used for programming RFID tag 178A on specimen slide 176A using RFID tag 174A on specimen block 170A. RFID reader 162 may read information from RFID tag 174A of block 170A, and write this information to RFID tag 178A of slide 176A and/or record the information as well as serial numbers of RFID tag 178A to SMS 4. For example, the information may include the case ID associated with specimen 180 on slide 176A. In a similar manner as described with respect to FIG. 8C, SMS 4 guides the user to program slide 176A as well as the other slides 176 for the patient case with information matching the corresponding block 170A from which specimen 180A originated. In other embodiments, SMS 4 may prompt the user to program one slide 176 from another slide 176. The same RFID tag format may be used for each of RFID tags 168, 174, and 178, or in other embodiments data may be contained on the tag to indicate the type of tag (e.g., bottle tag, block tag, or slide tag). RFID tags may be manufactured with the tag type already programmed. In other embodiments, some of bottles, blocks, or slides may include bar codes instead of RFID tags.

In some embodiments, RFID antenna pad 160 may include special form factors to hold bottles, blocks, slides, and combinations thereof. For example, RFID antenna pad 160 may include a pad with wells for bottles, slots for blocks, separate areas for blocks, bottles, and slides. RFID antenna pad 160 may be connected to network 6 (FIG. 1) and SMS 4 via a wireless connection, network cable, or may be connected to network 6 via client computing device 164. RFID reader 162 may include a processor, and can communicate over network 6 to a server for SMS 4. RFID antenna pad 160 and RFID reader 162 may be specially designed to withstand the laboratory environment. For example, RFID antenna pad 160 and RFID reader 162 may be waterproof or may include smooth surfaces for easy cleaning.

The programming function of SMS 4 may be triggered automatically as items are placed on RFID antenna pad 160, automatically based on another process running on client computing device 164, automatically when mixed item types are detected on RFID antenna pad, or may be initiated by a user via buttons associated with RFID reader 162 or a user interface of client computing device 164.

FIG. 8E is a block diagram illustrating an example RFID station 158 used for verifying accuracy of items for a patient case. RFID station 158 may be the same RFID station used for the programming process described with respect to FIGS. 8C and 8D. For example, the user may interact with system 158 for interrogating RFID tags 174A-174C (“RFID tags 174”) on specimen blocks 170A-170C (“blocks 170”) to verify accuracy of information programmed to RFID tags 174, and to verify that blocks 170 all correspond to a single patient case. Although shown with bottle 166 also present on RFID antenna pad 160, bottle 166 may or may not be used for the verification. The verification process may be performed immediately after blocks are programmed, as well as at other points during specimen management. SMS 4 may prompt the user to place blocks 170 one at a time onto RFID antenna pad 160 until all of the blocks 170 (or all that will fit) for a case are present on RFID antenna pad 160. Similar to the programming process, SMS 4 may tell the user specifically which blocks 170 to place on the pad sequentially in a predefined order for verification, and may provide alerts if a time period elapses before RFID antenna pad 160 detects the expected block 170, or if the wrong block 170 than was requested is detected. For example, FIG. 8E illustrates the state of RFID station 158 after the user has been prompted and placed the third block 170 onto RFID antenna pad 160.

FIG. 8F is a block diagram illustrating an example RFID station 175 used for verifying accuracy of items for a patient case. RFID station 175 may be the same RFID station used for the programming process described with respect to FIGS. 8C and 8D. For example, the user may interact with system 158 for interrogating RFID tags 178A-178D (“RFID tags 178”) on specimen slides 176A-176D (“slides 176”) to verify accuracy of information programmed to RFID tags 178, and to verify that slides 176 all correspond to a single patient case. Although shown with block 170A also present on RFID antenna pad 160, block 170A may or may not be used for the verification. The verification process may be performed immediately after blocks are programmed, as well as at other points during specimen management. SMS 4 may prompt the user to place slides 176 one at a time onto RFID antenna pad 160 until all of the slides 176 (or all that will fit) for a case are present on RFID antenna pad 160. Similar to the programming process, SMS 4 may tell the user specifically which slides 176 to place on the pad sequentially in a predefined order for verification, and may provide alerts if a time period elapses before RFID antenna pad 160 detects the expected slide 176, or if the wrong slide 176 than was requested is detected. For example, FIG. 8E illustrates the state of RFID station 158 after the user has been prompted and placed the third slide 176 onto RFID antenna pad 160.

Alternatively, FIGS. 8E and 8F may represent the RFID stations being used for programming RFID tags of the blocks or slides as a group. For example, multiple items (e.g., bottles, blocks or slides) may be programmed substantially simultaneously with the same information, such as a timestamp indicating a time the bottles arrived at the laboratory. In this case, the bottle 166 shown in FIG. 8E and the block 170A shown in FIG. 8F may or may not be present. Programming of the items may only be permitted when the complete set of items is detected at once, or is detected within a predefined time period. As another example, items may be programmed individually but without removing the items from the RFID antenna pad 160 after programming each one, such that the end result is that all of the items are present at the pad at once.

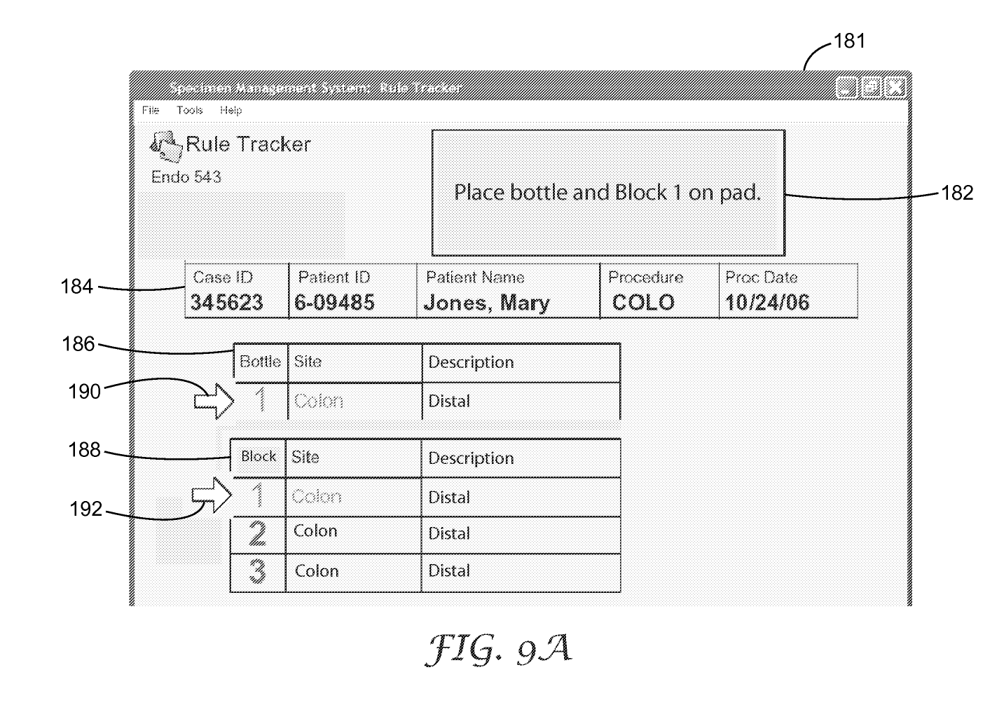

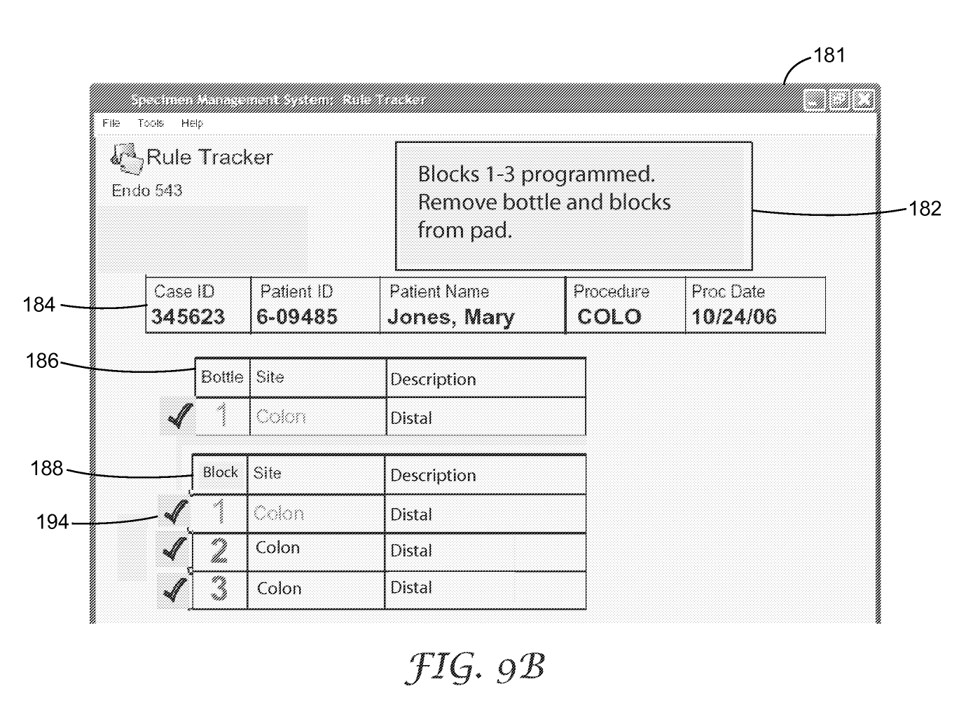

FIGS. 9A-9B are screen illustrations illustrating an example user interface 181 of the SMS 4 for programming RFID tags. For example, user interface 181 may be displayed on client computing device 164 of FIGS. 8C-8D. In the example of FIG. 9A, message window 182 instructs the user to place a bottle and Block 1 onto RFID antenna pad 160. Case entry line 184 indicates the present case being programmed. Bottle table 186 shows the current bottle to be placed on RFID antenna pad 160, highlighted by arrow 190. Block table 188 shows the blocks to be placed on RFID antenna pad 160, the first block highlighted by arrow 192. As each block is placed on RFID antenna pad 160 and programmed, arrow 192 may move down to highlight the next block.

In the example of FIG. 9B, message window 182 indicates that block 1 through 3 have been programmed for the case indicated in case entry line 184, and instructs the user to remove the bottle and the block from RFID antenna pad 160. Check marks 194 indicate that blocks 1-3 have been successfully programmed.

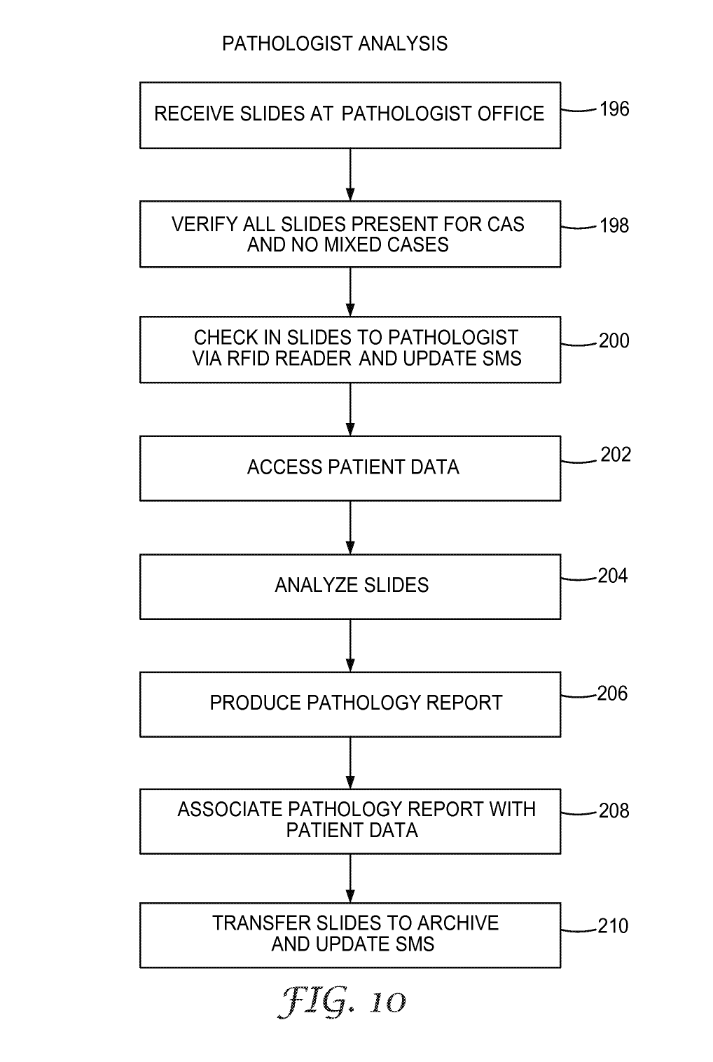

FIG. 10 is a flowchart illustrating in further detail an example process during analysis of patient specimens by a pathologist. Initially, slides 20 (FIG. 1) for a given case are received at pathologist office 24 (196). A user may initially verify that all of the slides 20 for the case are present and that no mixed cases occur (i.e., slides from more than one patient case) (198). The verification may be performed at an RFID station 42 within the laboratory as described in further detail below with respect to FIGS. 12 and 13A-13C. The user may also check in the slides to pathologist office 24 via an RFID reader 48 at the RFID station 42 (200). Checking in the slides may cause SMS 4 to create a new entry in SMS database 41 with a timestamp that indicates the slides were verified as present in laboratory 16 at the time indicated by the timestamp. At this time, SMS 4 may automatically access patient management system 9, LIS 22 or other system via one or more APIs and present patient data to the user based on the case ID obtained from the slides 20 or the patient record within SMS 4 (202). For example, the user may obtain patient records from patient management system 9, or dictation about the collected specimen (e.g., from the endoscopist who collected the specimen) from a dictation system. This eliminates the need to manually enter label information from the slides, which may be prone to data entry errors.

The pathologist may then analyze the slides, e.g., using a microscope (204), and produce a pathology report based on the analysis (206). SMS 4 may cause an alert to be provided when the pathologist's report is not generated within a given time period of receiving the slides at the pathologist's office. An RFID reader antenna may be designed to fit near the slide stage on the microscope, or may be integrated into the microscope, such that when a slide is placed on the microscope, the RFID tag on the slide is within an interrogation region of the RFID reader antenna and may be read and the appropriate patient information displayed by SMS 4. In one embodiment, the RFID reader/antenna may be incorporated into the stage area of the microscope. This may help further ensure that the pathology report is based on and associated with the proper patient's data record (208). Such a system may also indicate whether the user has viewed all of the slides for a case. Paperwork that accompanies the slides may also have an RFID tag affixed to it that may also be read by a reader on or near the microscope. When the pathologist is finished with the pathology report, the slides may be transferred to an archive location, and SMS database 41 updated to reflect the transfer (210).

In one embodiment, a user interface may simultaneously present to the user on a single screen a view of the slide in the microscope and the pertinent patient data obtained via the RFID-enabled SMS 4. Alternatively, the user interface of a client computing device may be positioned near to the microscope eyepiece at an angle that allows for comfortable viewing of both the user interface and the eyepiece.

In some embodiments, RFID tags may be used by the pathologist in entering information for a case. For example, in some areas of pathology there may be only a few main diagnosis options possible. Different pre-programmed RFID tags may represent the different diagnoses such that the pathologist can select an appropriate pre-programmed RFID tag and place the tag on the reader to indicate the determined diagnosis. In this way, a set of RFID tags preprogrammed with diagnosis codes may save time by eliminating data entry, and avoid confusion that may arise when hand-written notes are entered by clerical staff. SMS 4 may also provide a pathology audit trail, such that when a slide having an RFID tag is presented to a reader, SMS 4 can bring up a history of activity associated with the slide. This may include identification of who has looked at the slide, what diagnoses or comments were previously entered, and other information about the history of the slide. SMS 4 may also provide automatic alerts to alert the pathologist to pathology reports that were expected but not produced, or to identify high priority cases to help the pathologist manage and sequence his or her workload.

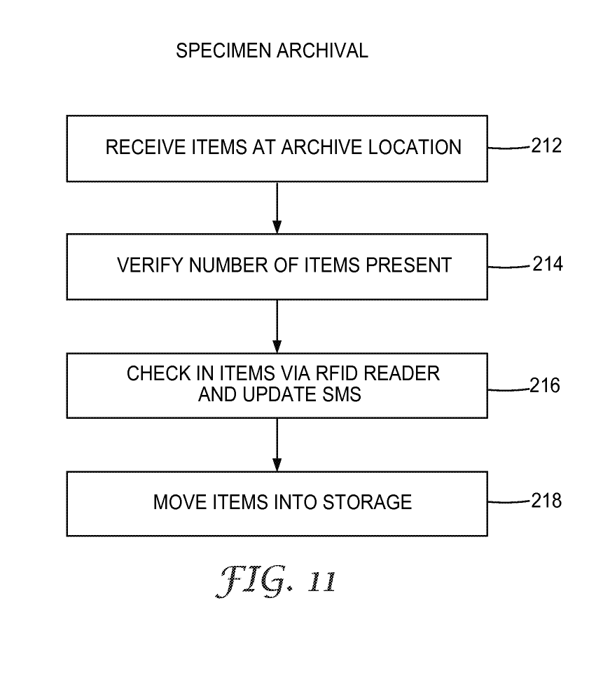

FIG. 11 is a flowchart illustrating in further detail an example process in which RFID tags and SMS 4 are used during archival of patient specimens. Items are received at archive 30 (212). A user may initially verify that all of the expected items for the case are present (214). The items may include any remaining bottles or blocks, and all of the slides. The verification may be performed at an RFID station 42 within archive 30 as described in further detail below with respect to FIGS. 12 and 13A-13C. The user may also check in the items to archive 30 via an RFID reader 48 at the RFID station 42 (216). Checking in the items may cause SMS 4 to create a new entry within the client record of in SMS database 41 with a timestamp that indicates the items are present in laboratory 16 at the time indicated by the timestamp. The items may then be moved into storage within archive 30 (218). The items may also be checked out of the archive at a later time.

FIG. 12 is a flowchart illustrating in further detail an example process for verification of accuracy of specimen information by SMS 4. FIG. 12 will be described with reference to FIGS. 13A-13C. FIGS. 13A-13C are screen illustrations illustrating an example user interface of SMS 4 for verification of accuracy of specimen information. As mentioned above, verification may be performed at multiple stations along the process from collection to analysis of specimens. For example, a verification routine may automatically occur after items are programmed, and may involve confirming that the right information was programmed to the right bottle, that a complete case was programmed, and that no mixed case is present. Verification may also occur at points along the process, such as when items are in transit, and may involve making sure that a complete set of items is present with no mixing of cases. The verification process will be described in terms of specimen bottles, but may similarly be applied in the context of blocks or slides.

Verification may take place at an RFID station 42. The user interface of client computing device 50 may prompt the user to place all bottles for a case onto the RFID antenna pad 44, or the user interface may prompt the user to place the bottles onto the RFID antenna pad 44 one at a time in a prescribed sequence within a time period until all bottles are on the pad (220). RFID antenna pad 44 detects RFID tag of the bottle placed on RFID antenna pad 44 (222). An RFID reader 48 at RFID station 42 may cumulatively retain information obtained from each of the sequentially interrogated RFID tags within a time period, and make the determination of whether a set of containers is a complete set based on the cumulatively retained information.

When RFID antenna pad 44 detects mixed bottles (i.e., bottles from two or more patient cases) (224), the user interface provides an alert to the user (226). The alert may be audible sound and/or a visual indication on client computing device 50. In the example of FIG. 13A, message window 240 of user interface 238 informs the user that mixed cases were detected, and instructs the user to place bottles from only a single case onto RFID antenna pad 44. The user can see from bottle table 246 that not all of the bottles have the same case ID and other information.

When RFID antenna pad 44 detects a blank tag (228), the user interface also provides an alert to the user (230). In the example of FIG. 13B, message window 240 indicates that a blank tag was detected. As shown in FIG. 13B, the user interface may also provide an alert when a tag has a case ID that is unknown, i.e., does not correspond to an entry in SMS database 41. Entries 248 and 250 illustrate an unknown ID and blank ID.

When RFID antenna pad 44 detects that not all of the bottles for the case have been detected (e.g., within a time-out period) (232), the user interface provides an alert to the user (234). The alert may indicate that certain items are missing from the expected number of bottles. As another example, the user interface may provide an alert when it has prompted the user to place a particular bottle onto RFID antenna pad 44, but a time-out expires before RFID antenna pad 44 detects the bottle. When RFID antenna pad 44 has detected a complete case of bottles, the user interface provides a verification message to the user that the presence of all of the bottles for the case have been detected. As shown in FIG. 13C, message window 240 indicates that a complete case was detected, and prompts the user to remove the bottles from RFID antenna pad 44. Check marks 252 appear to indicate that the bottles have been detected.

In addition to visual verification, the user interface may provide sounds that match the number of items detected. For example, when three bottles are placed on RFID antenna pad 44 and detected, a series of three audio cues may be sounded. Where a larger number of bottles exist for a single case, such as more bottles than will fit on RFID antenna pad 44 at once, SMS 4 may allow for several of the bottles to be presented at one time, and for the data from the first batch of bottles to remain on the screen for a time period after the batch is removed. In some embodiments, the verification process may be integrated as part of the process of programming the RFID tags. For example, after an item is programmed, the user may remove the item from RFID antenna pad 44, place the item back onto RFID antenna pad 44, and the information programmed on the RFID tag as well as corresponding database information may be displayed for the user to check. The fact that verification occurred may be recorded in the RFID tag history (e.g., in SMS database 41), and may include the identity of the user who performed the verification (such as by detecting an RFID tag on an ID badge of the user).

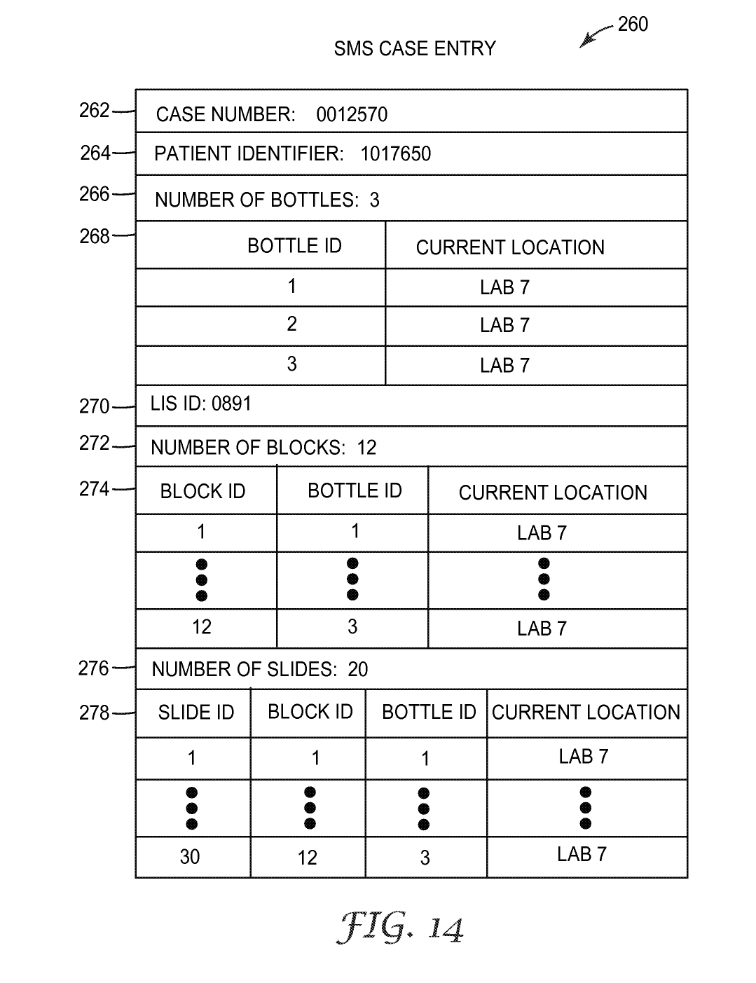

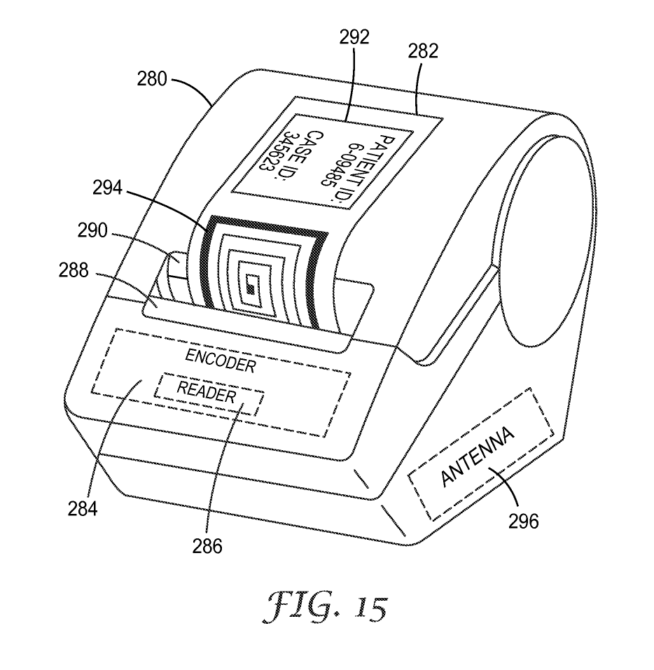

FIG. 14 is an example database entry within specimen management system 4, e.g., a case entry 260 in SMS database 41. Case entry 260 represents example data that may be maintained for a single case to track and link the numerous objects that may be used for conveying a patient's anatomic pathology specimens. As shown in FIG. 14, case entry 260 includes a case number field 262 and a patient identifier field 264, which may provide a reference to a patient record within patient management system 9. Bottle number field 266 indicates a number of bottles associated with the case, and bottle ID field 268 provides a listing of the unique identifiers for the RFID tags associated with each bottle and a current location of each bottle. An LIS ID field includes an LIS identifier, which provides a reference to a patient record within LIS 22.

Block number field 272 indicates a number of blocks associated with the case, and block ID field provides a listing of the unique identifiers for the RFID tags of the blocks, the ID of the RFID tag for the bottle from which the tissue block was obtained, and a current location of each block. Slide number field 276 indicates a number of slides associated with the case, and slide ID field provides a listing of the unique identifiers for the RFID tags of the slides for the patient, the IDs for the RFID tags for the block and bottle from which the tissue on the slide was obtained, and a current location of each slide. In the example of FIG. 14, all of the bottles, blocks, and slides are located in LAB 7 because the specimens are in laboratory 7 and have just been processed into blocks and slides.

Case entry 260 is merely exemplary; more or less information may be stored in SMS database 41 for a case. For example, case entry 260 may include a patient name field and a history log of previous locations and timestamps of items associated with the case. Case entry 260 may also include an intended destination field that indicates the destination for items associated with the case. If the item is tracked at an unexpected location (i.e., a location not on a usual path to the intended destination), an alert may be provided to the user along with information for getting the item back on the expected path.

SMS 4 may provide a system status report upon request by a user. The system status report may summarize any exceptions that have occurred (e.g., on a given day). For example, the system status report may indicate that 100 cases were processed yesterday, but that three of the cases had not yet been programmed (or, e.g., verified, accessioned, and the like). The system status report may enable the user to easily examine the information associated with the case, such as tracking history of items for the case.

SMS 4 may also provide alerts when specific events occur or fail to occur. The alerts may be in the form of a report, an email, a page to a pager, a call to a telephone or cellular telephone, or an electronic note on a computer desktop. Alerts may be initiated in a number of cases; for example: an item was sent from location A but not received at location B within a given time period; an item is missing; a pathology report has not been received within a time period; a specimen was collected but not entered into the tracking system; an anticipated next step in the process was not performed within a time period; and item was found at an unexpected location; a process step was skipped; or other events.

When an RFID tag of an item is read, the history of the item may be checked to ensure the item has been through a prescribed sequence of steps prior to the reading. An alert may be provided when any problems are identified. An alert may also be provided when a timestamp of a previous reading at a previous location occurs longer ago than a prescribed time period.

FIG. 15 is a block diagram illustrating a printer 280 that performs the steps of printing label 282 with label information 292, programming RFID information to RFID tag 294, as and verifying that the programmed RFID information is correct. Printer 280 includes a print output 290 for printing label information 292 to label 282. Label information 292 may be human-readable information, such as text or symbols, or may be machine-readable information, such as a bar code. Printer 280 also includes RFID encoder 284 embedded within printer 280. RFID encoder 280 programs RFID tag 294 with information, such as patient information, case information, specimen information, a unique ID, or other information, using RFID reader 286 and RFID antenna 288. As shown, RFID antenna 288 may be located proximate print output 290. RFID antenna 288 may have a short read range, such as approximately 1 inch (2.54 cm) or less. In this manner, RFID antenna 288 may be used by RFID encoder 280 to program only one RFID tag 294 from among a roll of labels 282, without programming any other RFID tags on the roll of labels.

Printer 280 also includes a second RFID antenna 296 coupled to RFID reader 286. Second RFID antenna 296 may have a longer read range, such as at least 4 inches (10.16 cm). Second RFID antenna 296 may be able to perform operations that RFID antenna 288 cannot perform because of the short read range of RFID antenna 288. For example, second RFID antenna 296 may be used for interrogating RFID tag 294 after label 282 has been printed/encoded and applied to an anatomical pathology specimen container (e.g, a bottle, block, or slide). Second RFID antenna 296 may verify that the information programmed to RFID tag 294 is accurate. Second RFID antenna 296 may alternatively or additionally be used to program RFID tag 294 with additional information not programmed by RFID antenna 288, such as information associated with a patient case or other information. Second RFID antenna 296 may program RFID tag 294 with information obtained from SMS 4.

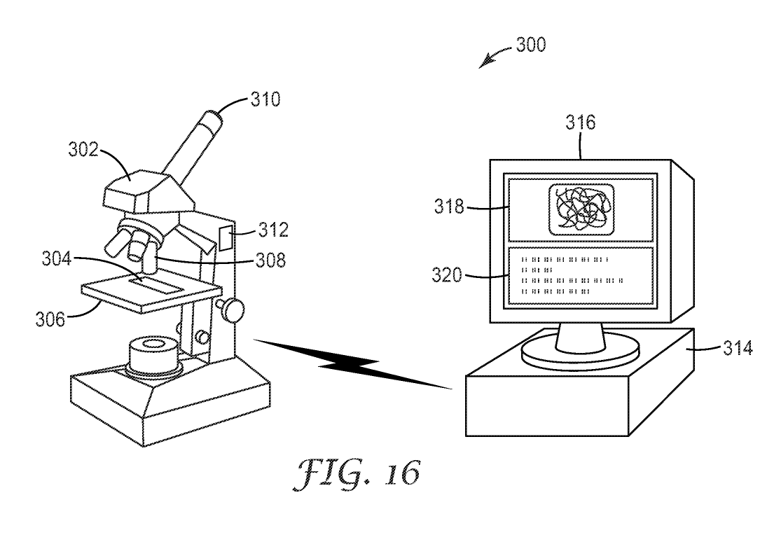

FIG. 16 is a block diagram illustrating an example system 300 having a microscope 302 with an RFID reader 312 for reading an RFID tag associated with a specimen slide 304. Microscope 302 includes a stage 306 for mounting a slide 304, and a lens 308 that magnifies the slide 304. Eyepiece 310 may be used for viewing a magnified view of slide 304. An RFID reader 312 may be attached to microscope 310 or integrated within microscope 310, and may be designed to be located near slide stage 306 of the microscope such that when slide 304 is placed on the microscope, the RFID tag on the slide 304 is within an interrogation region of an antenna of RFID reader 312 and may be read by RFID reader 312.

Microscope 302 may be in communication with client computing device 314. Reading the RFID tag of slide 304 may trigger the opening of a patient record by client computing device from SMS 4 or LIS 22, and cause the appropriate patient information to be displayed on display 316. Alternatively, if the patient record has already been opened, reading of the RFID tag of slide 304 can verify that the slide ID matches the ID of the open record, and provide an alert to the user if it doesn't match. This may help further ensure that the pathology report is based on and associated with the proper patient's data record. In addition reading the RFID tag of slide 304 may automatically cause the pathologist's report to be associated with the patient case based on information retrieved from the RFID tag.

In one embodiment, display 316 may simultaneously present to the user on a single screen a slide view 318 of slide 304 as magnified by microscope 302 and the pertinent patient data obtained via the RFID-enabled SMS 4 and displayed as a patient data view 320 on display 316. Alternatively, display 316 of a client computing device 314 may be positioned near to the eyepiece 310 of microscope 302 at an angle that allows for comfortable viewing of both display 316 and eyepiece 310. In another embodiment, client computing device 314 may transfer patient information obtained from SMS 4 to microscope 302, and eyepiece 310 may present to the user a combined view of both the magnified slide and the patient information.

Various embodiments of the invention have been described. For example, although described with respect to an environment for processing of anatomic pathology specimens, the techniques may also be employed in a variety of other environments, such as processing clinical specimens. As another example, although described for purposes of example with respect to RFID tags, bar codes may be used in addition or in the alternative. For example, items such as bottles, blocks, or slides may have bar code labels in place of RFID tags for tracking the items. These and other embodiments are within the scope of the following claims.

1. A printer comprising:
a print output for printing label information to a label;
a radio-frequency identification (RFID) encoder embedded within the printer for programming an RFID tag associated with the label produced by the print output, wherein the RFID encoder includes an RFID reader and a first RFID antenna; and
a second RFID antenna coupled to the RFID reader for reading information from the RFID tag, wherein the reader verifies accuracy of information programmed to the RFID tag on the label based on the information read from the RFID tag.

2. The printer of claim 1, wherein the first RFID antenna is located proximate the print output and has a read range of approximately 2.54 cm or less; and wherein the second RFID antenna has a read range of at least 10.16 cm.

3. The printer of claim 1, wherein the label information includes human-readable information.

4. The printer of claim 1, wherein the label information includes machine-readable information.

5. The printer of claim 1,
wherein the label is a label for application to an anatomical pathology specimen container, and
wherein, after application of the label to the anatomical pathology specimen container, the second RFID antenna verifies the accuracy of the information programmed to the RFID tag on the label by the first RFID antenna.

6. The printer of claim 1, wherein the second RFID antenna programs the RFID tag with information associated with a patient case.

7. The printer of claim 1, wherein the label is a label for application to an anatomical pathology specimen container, and wherein the second RFID antenna programs the RFID tag with information associated with a patient case maintained by an anatomical pathology specimen tracking system.

8. A method comprising:
printing label information to a label with a print output of a printer;
programming a radio-frequency identification (RFID) tag associated with the label produced by the print output with an RFID encoder embedded within the printer;
reading the RFID tag with a second RFID antenna coupled to the RFID reader; and
verifying the accuracy of information programmed to the RFID tag on the label based on the information read from the RFID tag.

9. The method of claim 8, further comprising applying the label to an anatomical pathology specimen container after printing the label information and programming the RFID tag.

10. The method of claim 8, wherein verifying the accuracy of the information programmed to the RFID tag comprises verifying after applying the label to the anatomical pathology specimen container.

11. The method of claim 8, wherein programming the RFID tag comprises programming the RFID tag with information associated with a patient case.

12. The method of claim 8, wherein the label is a label for application to an anatomical pathology specimen container, further comprising programming the RFID tag by the second RFID antenna with information associated with a patient case maintained by an anatomical pathology specimen tracking system.
